SEQUENCE LISTING (13 sequences)

SEQ ID NO: 1 - DNA, 1734 bp, Hepatitis C virus
atgggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcgtga

SEQ ID NO: 2 - protein, 577 aa, Hepatitis C virus
MGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 3 - protein, 18 aa, Hepatitis C virus
SAYQVRNSSGLYHVTNDC

SEQ ID NO: 4 - protein, 18 aa, Hepatitis C virus
SSGLYHVTNDCPNSSIVY

SEQ ID NO: 5 - protein, 5 aa, Artificial
  PEPTIDE SEQUENCE FOR MUTATIONAL INSERTION
TLFKQ

SEQ ID NO: 6 - protein, 5 aa, Artificial
  PEPTIDE SEQUENCE FOR MUTATIONAL INSERTION
TGVST

SEQ ID NO: 7 - protein, 5 aa, Artificial
  PEPTIDE SEQUENCE FOR MUTATIONAL INSERTION
HTVST

SEQ ID NO: 8 - protein, 5 aa, Artificial
  PEPTIDE SEQUENCE FOR MUTATIONAL INSERTION
LCLNT

SEQ ID NO: 9 - protein, 5 aa, Artificial
  PEPTIDE SEQUENCE FOR MUTATIONAL INSERTION
WCLNN

SEQ ID NO: 10 - protein, 5 aa, Artificial
  PEPTIDE SEQUENCE FOR MUTATIONAL INSERTION
FLFKQ

SEQ ID NO: 11 - protein, 5 aa, Artificial
  PEPTIDE SEQUENCE FOR MUTATIONAL INSERTION
WCLNM

SEQ ID NO: 12 - protein, 5 aa, Hepatitis C virus
SSGLY

SEQ ID NO: 13 - protein, 4 aa, Hepatitis C virus
SAYQ

(19) United States
(12) Patent Application Publication
(10) Pub. No.: US 2008/0286752 A1
(43) Pub. Date: Nov. 20, 2008

(54) Methods for the production of HCV, assaying HCV entry, and screening drugs and cellular receptors for HCV

(75) Inventor: Ila Singh, Tenafly, NJ (US)

Correspondence Address: WILMERHALE/NEW YORK, 399 PARK AVENUE, NEW YORK, NY, 10022, US

(73) Assignee: The Trustees of Columbia University in the City of New York, New York, NY (US)

(21) Appl. No.: 11/400,419

(22) Filed: Apr. 7, 2006

Related U.S. Application Data
(60) Provisional application No. 60/669,643, filed on Apr. 8, 2005.

(51) Int. Cl. C12Q 1/70; C12N 15/63

(52) U.S. Cl. 435/5; 435/455

(57) ABSTRACT

The invention provides cell culture methods that efficiently produce new infectious HCV virions where such methods are based on the unexpected finding that culturing cells at lower temperatures, i.e., from about 20° C. to about 34° C., enables efficient methods dependent upon HCV E1E2 mediated fusion. The invention also provides fusion assay methods that are robust and reliable because of, at least in part, specific pH conditions, and HCV E1 and E2 proteins that contain a dimerization domain. The present methods are useful for propagating infectious HCV, for improved diagnostics, drug screening and basic research efforts relating to HCV receptor binding, HCV entry (binding (attachment) and fusion), replication, virion assembly and release. In another respect, the present invention provides methods for detecting HCV E1E2 mediated fusion, and related methods for identifying drugs or other molecules that can inhibit HCV fusion and for identifying mutations that can inhibit HCV fusion.

This application claims priority to U.S. Ser. No. 60/669,643, filed Apr. 8, 2005, which is hereby incorporated herein by reference in its entirety.

The invention disclosed herein was made in part with U.S. Government support from the National Institutes of Health grant number R21DK062235. Accordingly, the U.S. Government has certain rights in this invention.

This disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety.

### BACKGROUND OF THE INVENTION

Hepatitis C virus (HCV) is the most common cause of nonalcoholic liver disease and the leading reason for orthotopic liver transplantation in the U.S. Further, a significant proportion of patients with hepatocellular carcinoma (HCC) are infected with HCV. More than 100 million people worldwide and nearly four million people in the U.S. are currently infected with HCV. HCV is transmitted through blood and blood products.

HCV is a member of the family Flaviviridae, which includes the flaviviruses, pestiviruses, hepaciviruses and the GB viruses. HCV is currently the sole member of the hepacivirus genus. HCV measures 30 to 60 μm and is an enveloped virus with a single-stranded, linear, positive-sense RNA genome approximately 9.6 kb in length. The HCV genome contains one large open reading frame (ORF) encoding a polyprotein precursor of approximately 3,000 amino acids. The polyprotein is cleaved co- and post-translationally by cellular and viral proteinases into at least ten different products (see FIG. 10). The structural proteins, C (core) and E1 and E2 (envelope glycoproteins), are contained at the N-terminus of the polyprotein, and are followed by p7 and the nonstructural proteins (NS) 2, 3, 4A, 4B, 5A and 5B.

Envelope proteins mediate viral binding to receptor(s) and fusion with host cell membrane. Various cellular molecules have been implicated in HCV binding to cell surfaces. These include, CD81, a molecule of the tetraspanin family, SR-B1, and low-density lipoprotein receptor (LDL-R). However, since all of these molecules (except LDL-R), are present on many different cell-types and HCV infects only human and chimpanzee liver cells, it is thought that a second, as yet uncharacterized receptor specific to these liver cells, is necessary. Such a receptor has not been identified yet.

HCV is believed to be endocytosed upon binding. In the low pH of the endosome, HCV envelope proteins undergo a conformational change that allows the viral envelope to fuse with the endosomal membrane, releasing the viral core into the cytoplasm. The core disassembles and the viral RNA is translated by host-cell ribosomes to generate the viral polyprotein, thus initiating the synthesis of progeny virus. The viral polyprotein is cleaved co- and post-translationally to generate various non-structural proteins that play a role in viral RNA replication, a single kind of capsid protein, and the three envelope proteins.

HCV envelope proteins are anchored to the membrane via a single C-terminal transmembrane domain and contain an N-terminal ectodomain. The transmembrane domains are essential for correct heterodimerization. The transmembrane domains for E1 and E2 contain signals for retention of the proteins in the endoplasmic reticulum. It is thought that the viral cores bud into the endoplasmic reticulum (ER), and the virus is transported outside the cell via the secretory pathway. More recently, it has been shown that a small fraction of E1 and E2 escapes the ER-retention mechanism and is expressed on the cell surface.

The structure of a few flavivirus envelope proteins has been determined by electron cryomicroscopy, and X-ray crystallography. They are classified as class II fusion proteins and HCV envelope proteins are thought to belong in this class. Class II fusion proteins differ in structure from the better characterized class I fusion proteins. Class I fusion proteins form a-helical trimers arranged in ‘spikes’ perpendicular to the viral membrane. They contain a stretch of hydrophobic amino acids near the N-terminus constituting the fusion peptide. The mechanism of fusion by this class of proteins is understood in detail, and several inhibitors are currently being tested for their ability to block fusion of influenza HA and HIV env, both class I proteins. In contrast, the class II fusion proteins have ectodomains that consist of β-sheets and lie parallel to the membrane. At neutral pH, the proteins form homodimers, with extensive lateral interactions with their neighbors, resulting in an icosahedral shell. Recently, the structure of post-fusion Dengue virus E molecule was determined. At low pH, an irreversible conformational change takes place that converts the dimers to trimers. The hydrophobic fusion loop that is normally buried in the protein, inserts into the target membrane. The structure of this post-fusion trimer resembles the class I protein trimers. However, it is important to note that structural data exists only for the neutral pH and the post-fusion conformations.

Attempts to study HCV replication in cell culture with clinical isolates or molecular clones have met little success. Various hepatoma cell lines, B-cell lines, T-cell lines, primary hepatocytes and peripheral blood mononuclear cells have been reported to support HCV replication as assayed by the detection of minus-strand RNA by reverse transcription polymerase chain reaction (RT-PCR). However, the low levels of replication and protein expression have not enabled classical genetic and biochemical analysis. To improve studies of HCV replication, HCV subgenomic replicons have been developed. But the subgenomic replicons are limited by the fact that they do not encode for the structural proteins. This led to the development of a cell-culture system that attempts to provide full-length HCV RNA replication through the use of HCV that possess cell-culture adapted mutations. Replication of genome-length dicistronic HCV RNA has also been shown in a human liver cell line when HCV RNA was dicistronically transfected together with selective markers. Although replication of the entire HCV genome has been observed along with properly processed HCV proteins, neither the cell-culture adapted HCV method nor the dicistronic method produced infectious virions. Thus, viral RNA replication and synthesis of fully processed viral proteins may not be sufficient to produce infectious virus.

To better study HCV assembly and infection, infectious HCV pseudoparticles or pseudotyped particles (HCV-pp) containing functional E1-E2 envelope protein complexes were developed. The HCV-pp consists of full-length E1 and E2 proteins and a retroviral core. Although the HCV-pp is infectious, its chimeric nature only allows for the study of HCV early entry. Another method for the study of attachment and fusion involves the use of chimeric E1/E2 ectodomain-VSV fusion proteins. However, this method failed to result in syncytium formation and relies on the assumption that ectodomain chimeras behave similarly to full-length E1 and E2. Therefore, a reliable method for studying HCV entry (attachment and fusion) is needed that is capable of using full length E1 and E2.

Lastly, there is a report purporting that infectious HCV can be produced in a three-dimensional radial-flow bioreactor. (Aizaki, H. et al., Virology (2003), 314: 16-25.) The paper reports that FLC4 cells can produce new infectious HCV virions after initial infection by clinical isolates or after initial transfection with full-length HCV genomes. The authors believe that the ability of FLC4 cells to produce infectious HCV could be due to (1) host factors present in FLC4 that are not present in other commonly used human hepatoma cell lines and (2) the three-dimensional aspects of a radial-flow bioreactor (RFB). Cells cultured in the RFB system purportedly maintain their polarity within a well-defined 3D structure having tight intercellular junctions and close connectivity with other epithelial cell membranes, such that the liver cells can maintain their physiological function for a long time in culture. However, the RFB system is still inefficient for the growth of HCV particles in culture compared to that of other known viruses. In the culture medium of FLC4 cells in RFB culture, quantitative RT-PCR only detected 106 to 107 copies/day of HCV RNA 2-3 weeks after infection, and only 10-106 copies/day of HCV RNA 2-4 months after transfection. Further, being a complicated three-dimensional device, the RFB system is not practical for standard screening and structure/function studies that depend on two-dimensional (plates) and simple three-dimensional (flasks) cell culture methods. A more efficient and robust method for producing infectious HCV is desired.

### SUMMARY OF THE INVENTION

The present invention relates to methods for (1) propagating infectious hepacivirus particles, including infectious HCV particles, (2) for detecting HCV envelope glycoprotein mediated membrane fusion, (3) for detecting or identifying mutations that affect HCV propagation or any step in the HCV life-cycle, including entry (binding and fusion), replication, assembly, release and infection, (4) for screening or identifying drugs that inhibit or prevent HCV propagation, including screening or identifying drugs that inhibit HCV at a particular step in the HCV life-cycle, including entry (binding and fusion), replication, assembly, and release, and (5) for detecting or identifying host proteins that are involved in HCV propagation or any step in the HCV life-cycle, including identifying host cellular receptors that mediate HCV virion entry (binding and/or fusion). The methods of the present invention are based in part on the unexpected finding that incubation of cells expressing full-length HCV E1 and E2 glycoproteins at low temperatures (Example 1) and/or low pH (Example 5) allows for robust and reproducible fusion. The growth or incubation of HCV at low temperatures and/or low pH allows for the efficient propagation of infectious hepacivirus particles and other methods that involve HCV particles, HCV nucleic acids or HCV proteins in cell-culture.

In the present invention, the temperature for incubating, culturing, growing, propagating or any other step that involves a low temperature cell-culture condition for HCV, refers to the air temperature of the compartment or device (i.e., “shelf temperature”) in which a cell culture is conducted. Low temperature cell culture conditions for HCV can comprise any sustained incubation of cells below 37° C. For example, low temperature cell culture conditions for HCV can comprise a temperature from about 20° C. to about 34° C., from about 20° C. to about 32° C., from about 25° C. to about 32° C., from about 28° C. to about 32° C., from about 25° C. to about 30° C., from about 26° C. to about 30° C., from about 29° C. to about 30° C., from about 25° C. to about 29° C., from about 28° C. to about 29° C., from about 25° C. to about 28° C., from about 25° C. to about 27° C., or from about 25° C. to about 26° C. It is understood by one skilled in the art that over time, the temperature of cell culture media and cells present in the media will be the same or substantially the same as the shelf or air temperature of the incubator in which the cells are cultured.

In one aspect, the invention provides a method for propagating infectious virus particles in a monolayer or suspension cell culture which comprises: (a) (i) infecting a cell with a virus particle comprising a hepacivirus E1 protein and a hepacivirus E2 protein or (ii) transfecting a cell with one or more nucleic acids comprising a hepacivirus nucleic acid, wherein the one or more nucleic acids encode a virus; and (b) incubating the cell at a temperature from about 20° C. to about 34° C. in an appropriate cell culture system, thereby propagating infectious hepacivirus particles. In one application of the aspect, the cell culture system is not a bioreactor. The cell culture system can comprise, for example, monolayers of cells grown and/or incubated on plates, suspensions of cells grown and/or incubated in tissue-culture flasks, or suspensions of cells grown and/or incubated in tissue culture roller drums. The one or more nucleic acids comprising a hepacivirus nucleic acid can comprise a sequence or sequences encoding a hepacivirus E1 protein and a hepacivirus E2 protein. The one or more nucleic acids can comprise a full-length hepacivirus genome, including a full-length hepatitis C virus genome. The full-length genome can be a wild-type or a mutant genome. The sequence or sequences encoding the hepacivirus E1 and E2 proteins can encode full-length hepacivirus E1 and E2 proteins or mutants, truncations or fusions thereof. For the virus particle comprising a hepacivirus E1 protein and a hepacivirus E2 protein, the hepacivirus E1 and E2 proteins can be full-length proteins, fusions or truncations, wild-type or mutant.

In one aspect, the invention provides a method for propagating infectious hepatitis C virus (HCV) particles in a monolayer or suspension cell culture which comprises: (a) (i) infecting a cell with a virus particle comprising an HCV E1 protein and an HCV E2 protein or (ii) transfecting a cell with one or more nucleic acids comprising a HCV nucleic acid, wherein the one or more nucleic acids encode a virus; and (b) incubating the cell at a temperature from about 20° C. to about 34° C. in an appropriate cell culture system, thereby propagating infectious HCV particles. In one application, the appropriate cell culture system does not include a bioreactor. In other applications, the appropriate cell culture system includes a bioreactor, but the incubation occurs in a bioreactor where the temperature is, for example, from about 20° C. to about 32° C., from about 20° C. to about 30° C., or from about 26° C. to about 30° C. The appropriate cell culture system comprises a monolayer or a suspension tissue culture system. A bioreactor can be a radial-flow bioreactor. The one or more nucleic acids comprising a HCV nucleic acid can comprise a sequence or sequences encoding a HCV E1 protein and a HCV E2 protein. The one or more nucleic acids can comprise a full-length HCV genome. The full-length genome can be a wild-type or a mutant genome. The sequence or sequences encoding the HCV E1 and E2 proteins can encode full-length HCV E1 and E2 proteins or mutants, truncations or fusions thereof. HCV E1 and E2 fusions can include chimeric fusions, including chimeric HCV E1 and E2 proteins that can form heterodimers. For the virus particle comprising a HCV E1 protein and a HCV E2 protein, the HCV E1 and E2 proteins can be full-length proteins, fusions or truncations, wild-type or mutant.

In the present invention, in relation to cells that are infected with hepacivirus or HCV particles or transfected with hepacivirus or HCV nucleic acids, the cell that is infected or transfected can comprise, for example, a hepatocyte cell, a monkey kidney cell, a chimpanzee cell, a porcine kidney cell, a baby hamster kidney cell, a murine macrophage cell, a human macrophage cell, a human peripheral blood leukocyte, a human adherent macrophage, an embryonic cell, a stem cell, or a transformed cell. The cells can originate from human or chimpanzee livers. The cell can be a primary cell or a cell line. The cell line can comprise a human hepatoma cell-line. The human hepatoma cell line can comprise, for example, Huh-7, Huh-7.5, PLC/PRF-5, FLC4, Hep3B, HepG2 or HepG2-CD81. In some applications of the methods for propagation, the cell comprises the Huh-7 or Huh-7.5 cell line.

In the present invention, a virus particle (or virion) can comprise a wild-type particle, a mutant particle, or a pseudotyped particle. Also in the invention, nucleic acids that are transfected into cells may comprise, for example, a full-length wild-type virus genome, a HCV subgenome, or a combination of expression constructs comprising HCV envelope glycoproteins and retroviral or lentiviral core proteins.

In another aspect, the invention provides a method for identifying a drug that inhibits HCV propagation comprising: (a) (i) infecting a cell with an HCV particle or (ii) transfecting a cell with one or more nucleic acids comprising an HCV nucleic acid, wherein the one or more nucleic acids encode a virus; (b) contacting the cell with a drug in a cell culture system; (c) incubating the cell culture system at a temperature from about 20° C. to about 34° C.; and (d) assaying the cell culture supernatant for the presence of infectious HCV particles, wherein the absence or reduction of infectious HCV particles compared to a supernatant from an uninfected or non-transfected cell culture, identifies the drug as a drug that inhibits HCV propagation. The cell that is infected or transfected can comprise, for example, a liver cell expressing CD81, HLDLr, hSR-B1, or combinations thereof. The liver cell can comprise, for example, a primary cell line derived from a hepatoma or a hepatocellular carcinoma, or established cell lines such as Huh-7, Huh-7.5, PLC/PRF/5, Hep3B, HepG2-CD81, FLC4 or JHH-4.

In the invention, methods for identifying drugs include high-throughput methods. Libraries of small molecules (including libraries of peptidomimetics), libraries of peptide drugs, or panels of antibodies, for example, can be tested in multi-plate well formats for high-throughput screening. The drugs can be tested for inhibiting HCV propagation by incubating candidate drugs with HCV particles and permissive cells; supernatants from individual wells can be tested in a high-throughput manner by testing for HCV RNA, HCV particles or proteins, cell lysis, or whether the supernatants can be used to infect new wells of cells. Drug candidates for inhibiting particular HCV functions, such as fusion, can also be tested for in a high-throughput manner. For fusion, the invention provides various aspects of fusion assays that are especially amenable for high-throughput screening. These fusion assays are further described in the Examples.

In another aspect, the invention provides a method for identifying a mutation that modulates HCV infection of cells comprising: (a) (i) infecting a cell with an HCV particle comprising a genome having at least one mutation or (ii) transfecting a cell with one or more nucleic acids comprising an HCV genome having at least one mutation; (b) incubating the cell in a cell culture system at a temperature from about 20° C. to about 34° C.; and (c) assaying the cell culture supernatant for the presence of infectious HCV particles, wherein the absence or reduction of infectious HCV particles compared to a supernatant from a cell culture infected or transfected with an unmutated HCV genome indicates that the mutation modulates HCV infection of cells. The cell that is infected or transfected can comprise, for example, a liver cell expressing CD81, HLDLr, hSR-B1, or combinations thereof. The liver cell can comprise, for example, a hepatoma or a hepatocellular carcinoma. The hepatoma or hepatocellular carcinoma can comprise, for example, the cell line Huh-7, Huh-7.5, PLC/PRF/5, Hep3B, HepG2-CD81, FLC4 or JHH-4.

In another aspect, the invention provides a method for detecting HCV E1 and E2 mediated cell fusion comprising: (a) co-culturing in a cell culture system an effector cell with a target cell, wherein the effector cell comprises an HCV E1 protein and an HCV E2 protein, wherein E1 and E2 are displayed on the effector cell surface; (b) incubating the cell culture system at a temperature from about 20° C. to about 32° C.; and (c) assaying the co-culture for syncytium formation, thereby detecting HCV E1 and E2 mediated cell fusion.

In the aspects of the invention that relate to HCV envelope glycoprotein mediated cell fusion, a step of co-culturing an effector cell and a target cell requires a transient exposure of these cells to low pH, where low pH can comprise from between about pH 5.0 to about pH 6.0. In one aspect, incubating or co-culturing an effector cell and a target cell comprises a pH from between about pH 5.0 to about pH 5.5. In one aspect, incubating or co-culturing an effector cell and a target cell comprises a pH from between about pH 5.0 to about pH 5.4. In another aspect, the pH is about pH 5.0. In another aspect, the pH is about pH 5.5. In another aspect, the pH is about pH 5.4. The pH refers to the pH of the cell culture media in which the effector and target cells are incubated or cultured in. When the effector and target cells are co-cultured at a pH from about 6.0 to about 7.0 or greater, no fusion between the cells occurs. A sufficient transient exposure can comprise, for example, from about 1 second to about 2 minutes. In one aspect, the exposure of the cells to low pH conditions comprises from about 30 seconds to about 1 minute.

In the aspects of the invention that involve HCV envelope glycoproteins, the glycoproteins can comprise E1, E2, or p7 proteins, or combinations thereof, such as E1, E2 and p7 or E1 and E2. The HCV E1 and E2 proteins can comprise, for example, full-length HCV E1 and E2 proteins, mutant HCV E1 and E2 proteins that comprise at least an ectodomain and a dimerization domain, or at least an ectodomain. Mutant E1 and E2 proteins include chimeric fusion proteins, which comprise E1 and E2 ectodomains. In one aspect, chimeric fusion proteins that comprise E1 or E2 ectodomains can further comprise a dimerization domain. The dimerization domain can be, for example, a dimerization domain from E1 or E2, or an Immunoglobulin heavy chain transmembrane and cytoplasmic domains.

In another aspect, the invention provides a method for detecting HCV E1 and E2 mediated cell fusion comprising: (a) co-culturing in a cell culture system an effector cell with a target cell, wherein the effector cell comprises: (i) a trigger that activates a reporter, (ii) an HCV E1 protein displayed on the effector cell surface, and (iii) an HCV E2 protein displayed on the effector cell surface, and wherein the target cell comprises the reporter; (b) incubating the cell culture system at a temperature from about 20° C. to about 34° C.; and (c) assaying the co-culture for reporter activity, wherein detection of reporter activity indicates HCV E1 and E2 mediated cell fusion.

A trigger is usually a protein that can bind to a cis-regulatory region, such as a promoter or enhancer, upstream of a reporter gene, thereby activating reporter gene expression. Examples of reporter genes include, but are not limited to, green fluorescent protein (GFP), enhanced GFP (eGFP), red fluorescent protein (RFP), the red fluorescent protein from coral (dsRed), yellow fluorescent protein (YFP), and luciferase. A trigger generally refers to any agent or substance that can cause the activation of some reporter. For example, a trigger can comprise a chemical that when in contact with another chemical (reporter), a reaction results that produces light that can be detected. The trigger can comprise an enzyme that cleaves a substrate such that the cleaved substrate acts as a reporter providing some kind of emission, such as fluorescence or chemilluminescence, etc. With respect to all aspects of the invention that involve a trigger that activates a reporter and a reporter, the trigger and the reporter can be in either the effector or target cell. If the trigger is in the effector cell, then the reporter is in the target cell. If the reporter is in the effector cell, then the trigger is the target cell.

In another aspect, the invention provides a method for detecting HCV E1 and E2 mediated cell fusion comprising: (a) co-culturing in a cell culture system an effector cell with a target cell, wherein the effector cell comprises: (i) a trigger that activates a reporter, (ii) a full-length HCV E1 protein displayed on the effector cell surface, and (iii) a full-length HCV E2 protein displayed on the effector cell surface, and wherein the target cell comprises the reporter; (b) incubating the cell culture system at a pH from about 4.8 to about 5.4; and (c) assaying the co-culture for reporter activity, wherein detection of reporter activity indicates HCV E1 and E2 mediated cell fusion. The effector cell can express E1 and E2, for example, from an expression vector comprising a polyprotein having the amino acid sequence of SEQ ID NO:2. E1 and E2 sequences can be derived from HCV genotypes 1a or 1b, and also from all other genotypes, such as 2a, 2b, 3, 4, 5 and 6. In one aspect, the target cell expresses high levels of CD81. In another aspect, the HCV E1 and E2 are not full-length but at least comprise an ectodomain and a transmembrane domain.

With respect to the methods that involve detection of fusion, the methods can comprise essentially any reporter system that comprises a two-part system, wherein one part is contained in the target cell and the second part is contained in the effector cell. Only when the two parts of the system come together, as in the cellular content mixing that occurs with cell-cell fusion, is there a measurable difference in reporter(s).

The reporter system for the fusion methods can also include essentially any fluorescent, chemilluminescent, or other system that emits signals that can be detected, as long as HCV glycoprotein dependent fusion between an effector and target cell can cause a detectable change in the signals. Thus, for example, even fluorescent membrane probes can be used in the present fusion assays. Fluorescent membrane probes can self quench at high concentrations and dequench when diluted, thereby emitting fluorescence. Thus, if the target (or effector) membrane contains the probe, it gets diluted upon fusion, thereby emitting fluorescence and indicating fusion. One of the oldest and still used examples of such probes is octadecylrhodamine B chloride (R 8).

In another aspect, the invention provides a method for identifying a polypeptide involved in HCV E1 and E2 mediated cell fusion comprising: (a) co-culturing in a cell culture system an effector cell with a target cell, wherein the effector cell comprises an HCV E1 protein and an HCV E2 protein, wherein E1 and E2 are displayed on the effector cell surface, and wherein the target cell comprises: (i) a polypeptide encoded by a library vector that is displayed on the target cell surface, and (ii) a CD81 protein displayed on the target cell surface; (b) incubating the cell culture system at a temperature from about 20° C. to about 34° C.; and (c) detecting increased syncytium formation as compared to a control co-culture, wherein the control co-culture has a target cell that does not comprise the library vector, wherein detection of increased syncytium formation indicates that the polypeptide is involved in HCV E1 and E2 mediated cell fusion. The target cell can further comprise an HLDLr protein displayed on the target cell surface or a SR-B1 protein displayed on the target cell surface, or both.

A method for identifying a polypeptide involved in HCV E1 and E2 mediated cell fusion comprising: (a) co-culturing in a cell culture system an effector cell with a target cell, wherein the effector cell comprises: (i) a trigger that activates a reporter, (ii) an HCV E1 protein displayed on the effector cell surface, and (iii) an HCV E2 protein displayed on the effector cell surface; and wherein the target cell comprises: (i) the reporter, (ii) a polypeptide encoded by a library vector that is displayed on the target cell surface, and (iii) a CD81 protein displayed on the target cell surface; (b) incubating the cell culture system at a temperature from about 20° C. to about 34° C.; and (c) assaying the co-culture for increased reporter activity as compared to a control co-culture, wherein the control co-culture has a target cell that does not comprise the library vector, wherein detection of increased reporter activity indicates that the polypeptide is involved in HCV E1 and E2 mediated cell fusion. The target cell can further comprise an HLDLr protein displayed on the target cell surface or a SR-B1 protein displayed on the target cell surface, or both.

In another aspect, the invention provides a method for identifying a host cell receptor involved in HCV binding and/or fusion into cells, the method comprising: (a) co-culturing an effector cell and a target cells at a temperature from about 20° C. to about 34° C., wherein the effector cell comprises (i) a native HCV E1, (ii) a native HCV E2, and (iii) a reporter, and wherein the target cell comprises (i) a non-hepatocyte cell, (ii) a polypeptide encoded by a library vector that is displayed on the target cell surface, wherein the library vector is from a hepatocyte-derived cDNA expression vector library, and (iii) a trigger that activates the reporter; (b) transiently incubating the co-culture at a pH from about 5.0 to about 6.0; and (c) assaying the co-culture for increased reporter activity as compared to a control co-culture, wherein the control co-culture comprises a target cell that does not comprise the expression vector, wherein detection of increased reporter activity indicates that the polypeptide is involved in HCV E1 and E2 mediated cell fusion.

In one aspect, the target cell can comprise a human or chimpanzee non-hepatocyte cell, which in their native state do not support fusion, but are capable of fusion when transfected with appropriate HCV receptors. Such a transfection can be performed with each individual candidate receptor gene, either by itself or in combination with others. Alternatively, target cells can be transfected with a hepatocyte-derived cDNA library, such that each target cell gets one, or in some cases, more than one, cDNA. In theory, one (or more than one) of those cDNAs will encode the HCV receptor(s). In case of target cells that have been individually transfected with candidate receptor genes, such monitoring can be under a microscope, looking for fluorescent cells, for example. Presence of fluorescent cells in numbers significantly above background will identify the said candidate receptor as likely playing a role in HCV entry. In case of target cells transfected en masse with a hepatocyte-derived cDNA library, the assaying for reporter activity (or selection based on reporter activity) can be performed by flow cytometry, sorting for cells expressing the reporter. These selected cells will contain the candidate receptor gene. The DNA from the selected cells can then be prepared and the candidate receptor identified by DNA sequencing.

In another aspect, the invention provides a method for producing HCV pseudotyped particles, the method comprising: (a) transfecting a cell with: (i) a plasmid comprising a coding sequence for native HCV E1 and E2 proteins, (ii) a plasmid comprising a coding sequence for retroviral Gag and Pol proteins, and (iii) a plasmid comprising a coding sequence for a fluorescent reporter protein and a retroviral packaging sequence; (b) incubating the transfected cell at a temperatures from about 20° C. to about 34° C.; and (c) harvesting HCV pseudotyped particles from a cell-culture media supernatant of the transfected cells. The HCV pseudotyped particles can then be used in the methods for identifying a host cell receptor involving in HCV binding and/or fusion. In step (b), the transfected cells are incubated at low temperatures in order to increase the efficiency of producing particles with HCV E1 and E2.

Thus, in another aspect, the invention provides a method for identifying a host cell receptor involved in HCV binding and/or fusion into cells, the method comprising: (a) incubating an HCV pseudotyped particle with a non-hepatocyte cell at a temperature from about 20° C. to about 34° C., wherein the HCV pseudotype particle comprises (i) native HCV E1 and E2, and (ii) a reporter, and wherein the non-hepatocyte cell comprises (i) a polypeptide encoded by a library vector that is displayed on the non-hepatocyte cell surface, wherein the library vector is from a hepatocyte-derived cDNA expression vector library, and (ii) a trigger that activates the reporter; and (b) assaying the non-hepatocyte cell for increased reporter activity as compared to a control non-hepatocyte cell, wherein the control non-hepatocyte cell does not comprise a library vector, wherein detection of increased reporter activity indicates that the polypeptide is involved in HCV E1 and E2 mediated cell fusion. In step (a), incubating the HCV pseudotyped particles with cells expressing the requisite HCV cellular receptors at low temperatures increases the efficiency of transduction, where transduction is dependent upon HCV glycoprotein mediated fusion. For pseudotype particles, a transient lowering of pH is not necessary because the particles are endocytosed and encounter low pH in the endosomes.

Again, the non-hepatocyte cells can be derived from non-hepatocyte human or chimpanzee cells, where these cells in their native state do not support fusion, but are capable of fusion when transfected with appropriate HCV receptors. Such a transfection can be performed with each individual candidate receptor gene, either by itself or in combination with others. Alternatively, cells can be transfected with a hepatocyte-derived cDNA library, such that each cell gets one, or in some cases, more than one, cDNA. In theory, one (or more than one) of those cDNAs will encode the HCV receptor(s). In case of transduced cells that have been individually transfected with candidate receptor genes, such monitoring can be under a microscope, looking for fluorescent cells, for example. Presence of fluorescent cells in numbers significantly above background will identify the said candidate receptor as likely playing a role in HCV entry. In case of transduced cells transfected en masse with a hepatocyte-derived cDNA library, the assaying (and selection) can be performed by flow cytometry, sorting for cells expressing the fluorescent reporter. DNA from selected cells with the candidate receptor gene can be prepared in order to identify the receptor by DNA sequencing.

A method for identifying a mutation that inhibits HCV E1 and E2 mediated cell fusion comprising: (a) co-culturing in a cell culture system an effector cell with a target cell capable of HCV E1 and E2 mediated cell fusion, wherein the effector cell comprises: (i) a reporter, (ii) an HCV E1 protein displayed on the effector cell surface, and (iii) an HCV E2 protein displayed on the effector cell surface, wherein at least the E1 protein or the E2 protein contains a mutation, and wherein the target cell comprises a trigger that activates the reporter; (b) incubating the cell culture system at a temperature from about 20° C. to about 34° C.; (c) transiently lowering the pH of the cell culture system to between about pH 5.0 and pH 6.0; and (d) assaying the co-culture for reporter activity, wherein no detection of reporter activity or detection of less reporter activity as compared to when E1 and E2 are not mutated indicates that the mutation inhibits or otherwise affects HCV E1 and E2 mediated cell fusion. In one aspect, the effector cell further comprises HCV p7. In relation to this aspect, a population of effector cells can be transfected with an expression library comprising E1 and/or E2 and/or p7 mutants. This method can be performed on each mutant of envelope glycoproteins individually in the co-culture, or on an entire library of mutants expressed en masse with the cell-specific expression of reporter allowing for a fusion read-out specific for each mutant.

A method for identifying a mutation that inhibits HCV E1 and E2 mediated cell fusion comprising: (a) co-culturing in a cell culture system an effector cell with a target cell capable of HCV E1 and E2 mediated cell fusion, wherein the effector cell comprises: (i) an active reporter, (ii) an HCV E1 protein displayed on the effector cell surface, and (iii) an HCV E2 protein displayed on the effector cell surface, wherein at least the E1 protein or the E2 protein contains a mutation, and wherein the target cell comprises a repressor of the reporter; (b) incubating the cell culture system at a temperature from about 20° C. to about 34° C.; (c) transiently lowering the pH of the cell culture system to between about pH 5.0 and pH 6.0; and (d) assaying the co-culture for reporter activity, wherein detection of reporter activity or detection of more reporter activity as compared to when E1 and E2 are not mutated indicates that the mutation inhibits or otherwise affects HCV E1 and E2 mediated cell fusion. In relation to this aspect, a population of effector cells can be transfected with an expression library comprising E1 and/or E2 mutants.

In one aspect, the invention provides a method for identifying a drug that inhibits HCV E1 and E2 mediated cell fusion comprising: (a) co-culturing in a cell culture system an effector cell with a target cell capable of HCV E1 and E2 mediated cell fusion, wherein the effector cell comprises: (i) a reporter, (ii) an HCV E1 protein displayed on the effector cell surface, and (iii) an HCV E2 protein displayed on the effector cell surface, and wherein the target cell comprises a trigger that activates the reporter; (b) adding a drug (or a peptide, protein, antibody, or any other molecule) to the cell culture system; (c) incubating the cell culture system at a temperature from about 20° C. to about 34° C.; (d) transiently lowering the pH of the cell culture system to between about pH 5.0 and pH 6.0; and (e) assaying the co-culture for reporter activity, wherein detection of no reporter activity or detection of less reporter activity as compared to when the drug is not added to the cell culture system indicates that the drug inhibits HCV E1 and E2 mediated cell fusion. In one aspect, the HCV E1 and E2 proteins are expressed in the effector cell from an expression vector encoding a polyprotein having the amino acid sequence of SEQ ID NO:2. In one aspect, the target cell expresses high levels of CD81.

In a variation of the fusion assay related aspects of the invention, any method that comprises a step of incubation, growth, infection, etc. at a temperature from about 20° C. to about 34° C., this step can be eliminated and replaced with a step of transiently lowering the pH (if the method does not already comprise the step of lowering pH) of the cell culture incubation, growth, infection, etc. to a pH that is from about 4.8 to about 5.4. In one aspect, the pH is from about 4.8 to about 6.2. In one aspect, the pH is from about 5.0 to about 5.4. In another aspect, the pH is about 5.4.

A method for identifying a drug that inhibits HCV E1 and E2 mediated cell fusion comprising: (a) co-culturing in a cell culture system an effector cell with a target cell capable of HCV E1 and E2 mediated cell fusion, wherein the effector cell comprises: (i) an active reporter, (ii) an HCV E1 protein displayed on the effector cell surface, and (iii) an HCV E2 protein displayed on the effector cell surface, and wherein the target cell comprises a repressor of the reporter; (b) adding a drug to the cell culture system; (c) incubating the cell culture system at a temperature from about 20° C. to about 34° C.; (d) transiently lowering the pH of the cell culture system to between about pH 5.0 and pH 6.0; and (e) assaying the co-culture for reporter activity, wherein detection of reporter activity or detection of more reporter activity as compared to when the drug is not added to the cell culture system indicates that the drug inhibits HCV E1 and E2 mediated cell fusion.

In the present invention, a target cell capable of HCV E1 and E2 mediated cell fusion can comprise, for example, cell-surface expression of CD81, cell-surface expression of CD81 and SR-B1, cell-surface expression of CD81 and HLDLr or cell-surface expression of CD81, SR-B1 and HLDLr. The target cell can comprise a cell of hepatocytic origin that expresses on its surface proteins that may function as HCV receptors. The exact nature of these receptors may or may not be characterized for the cells to be used as the target cell for fusion.

In the present invention, an effector cell can comprise, for example, full-length HCV E1 and E2 proteins such that the HCV E1 ectodomain and HCV E2 ectodomain are displayed on the cell surface and such that HCV E1 and E2 are dimerized, or HCV E1 and E2 chimera proteins that dimerize and which possess HCV E1 ectodomain and HCV E2 ectodomains that are displayed on the cell surface. The HCV glycoproteins can include p7, and can be of a sequence that occurs in a natural isolate, can contain changes introduced as a result of cloning, can contain mutations that may comprise point mutations, insertions, deletions or substitutions in the sequence, and can consist of chimeric structures containing parts from other genotypes or subtypes of HCV proteins or envelope proteins of viruses unrelated to HCV.

In the present invention, an appropriate cell culture system can comprise, for example, growing cells as monolayers, growing cells in suspension culture or in a bioreactor. However, when cells are grown in a bioreactor that comprise substructures, such as honeycomb-like substructures or porous bead microcarriers, where the substructures enable contact between cells and circulating components, then the invention contemplates the air temperature of the bioreactor, for example, to be from about 20° C. to about 32° C., from about 20° C. to about 30° C., from about 26° C. to about 30° C., from about 20° C. to about 28° C., or from about 26° C. to about 28° C. Otherwise, the low-temperatures of the invention for a bioreactor that is generally just a container in which a biological reaction (such as cell-growth or cell-maintenance or some other cellular phenomenon of cell culture incubation) takes place can comprise a temperature, for example, from about 20° C. to about 34° C., from about 20° C. to about 32° C., from about 25° C. to about 32° C., from about 28° C. to about 32° C., from about 25° C. to about 30° C., from about 26° C. to about 30° C., from about 29° C. to about 30° C., from about 25° C. to about 29° C., from about 28° C. to about 29° C., from about 25° C. to about 28° C., from about 25° C. to about 27° C., or from about 25° C. to about 26° C.

In one aspect, the invention provides a method for propagating infectious virus particles in a bioreactor cell culture which comprises: (a) (i) infecting a cell with a virus particle comprising a hepacivirus E1 protein and a hepacivirus E2 protein or (ii) transfecting a cell with one or more nucleic acids comprising a hepacivirus nucleic acid, wherein the one or more nucleic acids encode a virus; and (b) incubating the cell in a bioreactor cell culture at a temperature from about 20° C. to about 32° C., from about 20° C. to about 30° C., or from about 20° C. to about 30° C., thereby propagating infectious hepacivirus particles.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts a schematic of genetic footprinting. In the Figure, genetic footprinting is applied to identify particular mutations in HCV E1 and E2 that inhibits fusion. Mutants that do not allow fusion are not selected and sequenced. In comparison to the sequences before selection, one can determine by negative inference which regions are therefore important for fusion, i.e., mutations 3, 4 and 8 in FIG. 1. See also Example 4, and the insertional mutations discussed in Example 5 and presented in FIG. 11.

FIG. 2 depicts the basic scheme of the fusion assay of the present invention (see Example 1 and Example 5). In the figure, the “effector cell” is the 293T cell, which contains a green fluorescence protein (GFP) reporter gene under control of a T7 promoter and full-length HCV E1 and E2 proteins expressed at the cell surface. Here, the “target cell” is the Huh7 cell, which expresses on its cell surface receptors that can bind to E1 and E2 and which expresses T7 polymerase intracellularly. When the effector cell and the target cell are incubated together at low pH and low temperature, robust and reliable fusion is observed, either by microscopy (showing syncytium formation) or by detection of GFP fluorescence. In the assay, GFP will only be expressed when the target cell and the effector cell fuse, through the interaction of E1 and E2 with target cell receptors. Because the effector cell contains a plasmid that can express GFP via a T7 promoter, and the target cell expresses T7 polymerase, GFP expression occurs only upon fusion of the two cell types. Such a reporter system could consist of other fluorophores besides GFP or other chromophores.

FIG. 3A shows the microscopic results of a cell-cell fusion assay of the invention. 293T effector cells expressing viral envelope proteins (VSV-G in panel 1 on top, and HCV E1 and E2 in panel 2) were co-cultivated with target Huh-7 cells. Effector cells also contained a plasmid with a GFP gene under the control of the T7 promoter. Target cells were infected with the vaccinia virus producing T7 polymerase. Lowering the pH transiently resulted in formation of green multinucleated cells (cells on left). Background expression level of T7-GFP is seen in the empty vector control (panel 3). In the middle are immunofluorescence images, stained with the indicated anti-envelope antibody and Texas-red conjugated secondary antibody, showing that the multinucleated cells do indeed express the appropriate viral envelope protein. In case of the HCV panel, some single cells are also seen, indicating that the fusion process is not as efficient as for VSV-G. Also, note that the HCV syncytia are small in contrast to VSV-G induced syncytia. On the right are phase contrast images of the same cells. Panel 4 at the bottom shows infection (green) and transfection (red) controls. Here cells were transfected with T7-GFP and RFP plasmids and were subsequently infected with the vaccinia virus producing T7 polymerase. Each transfected cell appears red and each cell that was transfected and subsequently infected appears green. This green is due to fusion-independent expression of GFP. Note the size of these single cells as opposed to larger multinucleated cells above. Some size variation due to differences in sizes of 293T cells and Huh7 cells is also seen. FIG. 3B Green syncytia were seen following fusion mediated by HCV E1 and E2 (panel 1 on top) and VSV-G (panel 3) at low pH (see Example 5). Empty vector control is shown in panel 2. On the left of each panel are fluorescent images of the cells showing the GFP expressed upon fusion. In the middle are immunofluorescence images of the same cells (permeabilized and stained with anti-E2 mAb H52), showing that the syncytia do indeed express the appropriate viral envelope protein. On the right are phase contrast images of the same cells. Control cells (panel 4) were co-transfected with T7-GFP plasmid and expression construct for RFP, were subsequently infected with vaccinia virus producing T7 polymerase to show both fusion-independent expression of GFP and efficiency of vaccinia infection (green) and efficiency of transfection alone (red). Note the size of these single cells as opposed to the larger syncytia above. Some size variation due to differences in sizes of the two cell-types, 293T and Huh 7.5, is also seen. FIG. 3C presents close-up views of six syncytia resulting from fusion of cells expressing HCV envelope proteins.

FIG. 4 shows the microscopic results of an infection or transduction of Huh7 cells with pseudoparticles containing full-length HCV E1 and E2 proteins (see Example 2). The top left panel shows that the cells were successfully transduced with the pseudoparticles because staining with dsRed indicates that the retroviral genome from the pseudoparticles was present in the cells. Other constructs that served as a negative control yielded no transduction, of which a representative field is shown in the bottom panels.

FIG. 5 shows a proposed model of HCV E2 showing location of various mutations created in the HCVE2-VSV-G chimeric protein. Blue spheres represent mutants that are transported to the cell surface (1607, 1610, 1550, 1619, 1690, 2321, 2032) and red spheres represent mutants that are retained in the ER or degraded (2005, 1999, 1880, 2234, 2143, 2150) (see Example 3).

FIG. 6 shows a scheme for random mutagenesis using transposase (see Example 3).

FIG. 7 shows a scheme for selection of mutants using pseudotyping (see Examples 2 and 3).

FIG. 8 depicts a representative model for the genomic organization of HCV.

FIG. 9 shows a model of the complete HCV replication process or lifecycle.

FIG. 10 presents characteristics of fusion mediated by HCV envelope proteins. Fusion mediated by HCV E1 and E2 proteins (C22E1E2, genotype 1a) with Huh-7.5 target cells at pH 5.0 was taken as 100% and typically consisted of several hundred syncytia per well. Graphs represent the average of 3-4 independent experiments. FIG. 10A shows the efficiency of HCV E1E2 mediated fusion at different pH. Fusion at pH 5.0 was set to 100%. FIG. 10B shows the target cell specificity for fusion. FIG. 10C shows results using different expression constructs for E1 and E2 in the fusion assay. C22E1E2(1a) (with 22 amino acids of the C protein and the entire E1E2 sequences from HCV genotype 1a) and E1-VSVG+E2-VSVG contained a equimolar mix of two chimeric constructs, each containing the ectodomains of the HCV E1 or E2 protein (genotype 1b) along with the signal sequence, transmembrane domains and cytoplasmic tails of VSV-G protein. FIG. 10D shows the inhibition of HCV E1E2 mediated fusion using anti-HCV antibodies, and FIG. 10E shows the inhibition of fusion using peptides contained in HCV E1 sequences. Results were graphed as a fraction of fusion obtained in the absence of serum or peptides. For all experiments, except in FIG. 10A, data points were normalized by subtracting the amount of fusion that occurred in the control well treated with neutral pH fusion buffer. In FIG. 10A, this neutral pH control is seen on extreme right.

FIG. 11 presents a schematic map with the location of insertional mutations in HCV E1 and E2. The E1 and E2 envelope proteins of HCV genotype 1a, with the C-terminal 22 amino acids from the C protein, were used in Example 5. The amino acid sequence for this protein (SEQ ID NO:2) is listed in FIG. 14B, wherein residues 2-23 are the C-terminal 22 amino acids from the C protein, residues 24-215 are E1, and residues 216-577 are E2. The nucleotide sequence coding for the protein is SEQ ID NO:1 and is presented in FIG. 14A. Insertional mutants are designated by the position of the amino acid located towards the N-terminal end of the insertion (1 is the first methionine of the HCV polyprotein, C=1-192, E1=193-383, and E2=384-746; with respect to SEQ ID NO:2, 384 corresponds to residue 216 of SEQ ID NO:2). Proteolytic cleavage sites between C and E1, and between E1 and E2 are marked by arrows. Regions of focused mutant analysis are indicated on the map: HVR1=hypervariable region 1, TMD=transmembrane domain of E2, CD81=binding site for CD81.

FIG. 12 presents the results functional analysis results of some insertional HCV E1 and E2 mutants. FIG. 12A shows a cell-cell fusion assay performed with mutant E1E2 proteins. Cells expressing mutant E1E2 proteins were fused and processed for immunofluorescence. Fusion-competent mutant E2-388 resulted in green syncytia (top panel), and these expressed HCV E1 and E2 (central). Many cells expressing high amounts of envelope proteins did not form syncytia, as they were not adjacent to receptor expressing target cells. Mutant E2-545 (bottom panel) did not result in fusion, though it expressed abundant amounts of envelope proteins. FIG. 12B shows pseudoparticle infectivity. Pseudotyped particles were generated using the same mutants as shown in FIG. 12A. Particles were harvested, their amounts normalized and used to transduce Huh-7.5 cells. Successful transduction resulted in expression of DsRed 3-5 days later.

FIG. 13 presents a quantitative analysis of mutations in the hypervariable region, CD81-binding region and the transmembrane domains of HCV envelope proteins. Fusion activity of each mutant as compared to that of wild type E1E2, at pH 5.0 is shown in top panel. Results are from three separate experiments. IIF indicates production of envelope proteins by mutants, as visualized by indirect immunofluorescence, using polyclonal sera in permeabilized cells. Closed circles indicate that amount and localization of signal was indistinguishable from that produced by wild type proteins. Open circle indicates background staining. PP: Pseudoparticle assays with the same mutants. A ‘+’ indicates that the pseudotyped particles generated using the HCV E1E2 glycoproteins from that mutant were successful in transducing Huh-7.5 cells, as seen by Ds-red expression. All pseudoparticle preparations were normalized for reverse transcriptase activity before inoculation.

FIG. 14A presents the nucleotide sequence (SEQ ID NO:1) coding for the polyprotein presented in FIG. 11. FIG. 14B presents the amino acid sequence (SEQ ID NO:2) of the polyprotein schematically presented in FIG. 11. The coding sequence can be subcloned into, for example, any expression vector for use in the present methods. Although the coding sequence presented here is of HCV genotype 1a, one skilled in the art understands that any HCV sequence can be used such that a polyprotein can be designed comprising a methionine, a variable number of residues from the C-terminus of the C protein, the residues for E1, and the residues for E2.

### DETAILED DESCRIPTION OF THE INVENTION

The field of HCV research lacks a reliable and practical method for propagating infectious HCV such that structure/function and drug screening studies can be conducted in the context of the full HCV life-cycle. In one aspect, the present invention provides cell culture methods that efficiently produce new infectious HCV virions, thereby allowing the understanding of each step in the virus life cycle, which enables the design of protective vaccines, effective therapy and HCV diagnostics. Specifically, the present methods for propagating infectious HCV allow for improved diagnostics, drug screening and basic research efforts relating to HCV receptor binding, HCV entry (binding (attachment) and fusion), replication, virion assembly and release. In another respect, the present invention provides methods for detecting HCV E1/E2 mediated fusion, and methods for identifying drugs that can inhibit HCV fusion and therefore entry and for identifying mutations that can inhibit HCV fusion.

A key feature of the present methods (except for fusion assays as described in Example 5) is based on the unexpected finding that culturing applicable cells at lower temperatures, i.e., below 37° C., for example, from about 26° C. to about 32° C., enables efficient methods based around HCV E1/E2 mediated fusion. Because fusion is a key step in the virus lifecycle, this finding makes it reasonable to suspect that efficient methods for propagating infectious HCV virions may be dependent on culturing applicable cells at lower temperatures. The present methods based on the propagation of infectious HCV virions are more efficient than prior methods because culturing at lower temperatures allows for the production of levels of infectious HCV virions that are easily detectable, which are not masked by the release of HCV RNA-containing structures that lack the structural proteins. The present methods based on HCV E1/E2 mediated fusion are more efficient than prior methods because culturing at lower temperatures allows syncytium formation from successful fusion. The present methods based on HCV E1/E2 mediated fusion are more efficient that prior methods because culturing at lower temperatures allows more efficient fusion. Without being bound by theory, the culturing of HCV infected or transfected cells at lower temperatures enables the proper folding of HCV viral proteins, including E1 and E2 (full-length and fusion proteins of domains thereof), allowing for efficient propagation of infectious virions and for efficient fusion.

### Terms

As used herein, a “virion” refers to a structurally complete or mature virus, a viral particle.

As used herein, a “virus” refers to any of a number of small, obligatory intracellular particles with a single type of nucleic acid, either DNA or RNA. The nucleic acid is enclosed in a structure called a capsid, which is composed of repeating protein subunits called capsomeres, with or without a lipid envelope. The complete infectious virus particle, called a virion, must rely on the metabolism of the cell it infects. Viruses are morphologically heterogeneous, occurring as spherical, filamentous, polyhedral, or pleomorphic particles. They are classified by the host infected, the type of nucleic acid, the symmetry of the capsid, and the presence or absence of an envelope.

As used herein, a virus is “infectious” where viral replication occurs and virion progeny are released, or in other words, virus infection of a cell is productive of the generation of new virions, some of which are capable of infecting other cells and themselves generating new virions, of which some are infectious. This can be distinguished with viral infections that are abortive, in that cells are non permissive for a viral function and virion particles not produced, or with viral infections that are restrictive, where the infected cell is transiently permissive and a few virions are produced.

As used herein, an HCV “envelope glycoprotein” or “envelope protein” refers to HCV proteins E1, E2 and p7. Thus, in embodiments that comprise HCV E1 and E2, the embodiment can further comprise p7.

As used herein, a “bioreactor” is not simply a container in which a biological reaction takes place. Rather, the term “bioreactor” refers to containers that comprise substructures, such as honeycomb-like substructures or porous bead microcarriers, where the substructures enable contact between cells and circulating components.

As used herein, temperatures for cell-culture conditions refer to the shelf-temperature of an incubator or machine that is used to incubate or grow cells and tissues described in the present invention. It is understood by one skilled in the art that over time, the temperature of cell culture media and cells present in the media will be the same or substantially the same as the shelf or air temperature of the incubator in which the cells are cultured.

In certain embodiments, a “reporter” refers to a reporter system for detecting whether HCV glycoprotein mediated fusion occurs. With respect to the fusion methods of the invention, the methods can comprise essentially any reporter system that comprises a two-part system, wherein one part is contained in the target cell and the second part is contained in the effector cell. Only when the two parts of the system come together, as in the cellular content mixing that occurs with cell-cell fusion, is there a measurable difference in reporter(s). The reporter system can comprise essentially any fluorescent, luminescent (chemilluminescent, bioluminescent, etc.), or other system that emits signals that can be detected, as long as HCV glycoprotein dependent fusion between an effector and target cell can cause a detectable change in the signals. Thus, for example, even fluorescent membrane probes can be used in the present fusion assays. Fluorescent membrane probes can self quench at high concentrations and dequench when diluted, thereby emitting fluorescence. Thus, if the target (or effector) membrane contains the probe, it gets diluted upon fusion, thereby emitting fluorescence and indicating fusion. One of the oldest and still used examples of such probes is octadecylrhodamine B chloride (R18).

Thus, as used herein, a trigger generally refers to any agent or substance that can cause the activation of some reporter. A trigger is usually a molecule that can bind to a cis-regulatory region, such as a promoter or enhancer, upstream of a reporter gene, thereby activating reporter gene expression. Examples of reporter genes include, but are not limited to, green fluorescent protein (GFP), enhanced GFP (eGFP), red fluorescent protein (RFP), the red fluorescent protein from coral (dsRed), yellow fluorescent protein (YFP), and luciferase. Also, a trigger can comprise a chemical that when in contact with another chemical (reporter), a reaction results that produces detectable light. The trigger can comprise an enzyme that cleaves a substrate such that the cleaved substrate acts as a reporter providing some kind of emission, such as fluorescence or chemilluminescence, etc. In certain aspects of the invention, the reporter is a GFP gene under the transcriptional control of a T7 promoter and the trigger is a T7 polymerase protein. The T7 polymerase protein specifically binds to the T7 promoter and initiates transcription of the GFP gene. Transcription of the GFP gene leads to the production of GFP mRNA and translation of this RNA leads to production of GFP. Upon excitation by light, GFP emits fluorescence that is detectable. With respect to all aspects of the invention that involve a trigger that activates a reporter and a reporter, the trigger and the reporter can be in either the effector or target cell. If the trigger is in the effector cell, then the reporter is in the target cell. If the reporter is in the effector cell, then the trigger is the target cell.

In the present invention, reporter gene expression can serve as an indication as to whether a particular mutation or drug inhibits HCV propagation or HCV function at particular stages of its life-cycle, such as at the fusion step of cell entry. It is understood by one skilled in the art that reporter systems, such as luciferase and GFP provide background signal emission of light or fluorescence. Therefore, when the invention describes that a particular mutation or drug may cause “detection” or “no detection” of reporter activity, one skilled in the art understands that reporter activity may not be completely on or off. One skilled in the art understands that reporter activity is compared to negative and positive controls. For example, with respect to certain embodiments of the present fusion assays, an effector cell can comprise a GFP reporter gene construct under the control of a T7 promoter and HCV E1 and E2 proteins. A target cell can comprise T7 polymerase that binds to the T7 promoter and initiates GFP transcription such that GFP is expressed. Effector and target cells that fuse via HCV E1 and E2 binding to target cell receptors allow for the T7 polymerase to bind to the T7 promoter. Fluorescence from GFP can be detected by fluorescence readers, and the GFP fluorescence can be quantitated by such readers. A negative control may comprise of a system identical to the one described above, except that the effector cells are transfected with an ‘empty vector’ instead of one that expresses HCV E1 and E2. Such an empty vector contains all sequences in the expression vector except those that code for HCV E1 and E2. Such controls are important because in any individual population of effector and target cells that are tested for fusion, there is a percentage of effector and target cells that may not fuse (or may not be prevented from fusing with respect to testing mutations in E1 or E2 or with respect to testing drug inhibitors of fusion). Therefore, each experiment should be compared to controls to assess whether a mutation, a drug, or other variable causes an increase or decrease in fluorescence intensity with respect to negative and positive controls.

Prior methods for culturing HCV virions containing full-length genomes were either abortive, restrictive or so inefficient that the percentage of new virions produced that were themselves infectious was minimal. Thus, prior methods have attempted to circumvent these problems by using pseudotyped viruses that incorporate unmodified E1 and E2 onto retroviral cores. Pseudotype viruses incorporate full-length or unmodified E1 and E2 into retroviral cores, but do not possess all of the HCV proteins, which therefore do not allow a true study of the HCV lifecycle. In contrast, the present method liberates the production and study of HCV by enabling efficient cell culture methods that can use full-length E1 and E2, even in the context of the full-length HCV genome. Further, the present methods provide more efficient methods using pseudotyped viruses or other methods that rely on HCV glycoproteins due to, in certain embodiments, the use of low temperatures taught by the invention.

As used herein, the term “peptidomimetic” refers to a compound containing ono-peptidic structural elements that is capable of mimicking or antagonizing the biological action (s) of a natural parent peptide. Peptidomimetic compounds can mimic the critical features of the molecular recognition process of the parent peptide and thereby block or reproduce the action of the peptide. Peptidomimetics can in this definition thus be either antagonists or agonists. Probably the oldest example of a non-peptide peptidomimetic agonist for a peptide receptor system is morphine, which mimics the opioid peptides. As it relates to the invention, peptidomimetics can mimic the binding of HCV glycoproteins to cellular receptors or vice versa. In this manner, peptidomimetics can inhibit HCV infection by blocking virion attachment, entry or fusion.

### The HCV Virion

The HCV capsid protein (C) and the envelope glycoproteins are components of the HCV viral particle or virion. The HCV genome resides within the envelope of the HCV particle. The nonstructural proteins are not incorporated into the viral particle but are involved in the virus lifecycle, e.g., for HCV genome replication.

### HCV Genotypes, Subtypes and Isolates

The present methods are applicable to any hepacivirus, including HCV clinical isolates, cloned genomes and mutants. There are several major HCV genotypes, each of which has subtypes. The table below lists the HCV genotypes and subtypes, and how the current classification corresponds to old designations. The present invention encompasses all HCV genotypes and subtypes, including mutants, variants or homologues thereof. Specific sequences of the HCV genotypes and subtypes can be obtained from publicly available databases, such as the HCV sequence database, at http://hcv.lanl.gov.

TABLE 1
HCV Genotypes and Subtypes

| HCV genotype | HCV subtypes | Corresponding Old Designation |
|---|---|---|
| 1 | a, b, c, d, e, f, g, | current 1a = old “I” |
| | h, i, j, k, l, m | current 1b = old “II” |
| 2 | a, b, c, d, e, f, g, | current 2a = old “III” |
| | h, i, k, l, m | current 2b = old “IV” |
| 3 | a, b, c, d, e, f, g, | current 3a = old “V” |
| | h, i, k | current 3b = old “VI” |
| | | current 3k = old “10a/TD3” |
| 4 | a, b, c, d, e, f, g, | current 4n = old “4 alpha” |
| | h, k, l, m, n, o, p, | current 4o = old “4 beta” |
| | q, r, s, t | |
| 5 | a | — |
| 6 | a, b, d, f, g, h, i, | current 6c = old “7d” |
| | j, k, l, m, n | current 6d = old “7b” |
| | | current 6e = old “7a” |
| | | current 6f = old “7c/NGII/VII” |
| | | current 6g = old “11a” |
| | | current 6h = old “9a” |
| | | current 6i = old “9b” |
| | | current 6j = old “9c” |
| | | current 6k = old “8b” |
| | | current 6l = old “8a” |
| | | current 6n = old “NGI” |

At least six different genotypes and >90 subtypes of HCV exist. Approximately 70% of HCV-infected persons in the United States are infected with genotype 1, with frequency of subtype 1a predominating over subtype 1b. Different nucleic acid detection methods are available commercially to group isolates of HCV, based on genotypes and subtypes.

Nucleic acid and amino acid sequences for HCV subtypes can be obtained from sequence databases such as Genbank. For example, a non-limiting list of Genbank accession numbers for some HCV subtype sequences are: HCV subtype 1a [M62321] (HCV-1); subtype 1b [D90208] (HCV-J); subtype 2a [D00944] (HCV-J6); subtype 2b [D01221] (HCV-J8); subtype 3a [D17763] (HCV-NZL1); subtype 10a [D63821] (HCV-JK049); subtype 4a [Y11604] (HCV-ED43); subtype 5a [Y13184] (HCV-EVH1480); subtype 6a [Y12083] (HCV-EUHK2); and subtype 1a [D63822] (HCV-JK046).

### Genomic Organization of HCV and Functions of Viral Proteins

Based on its genome structure and virion properties, HCV has been classified as a separate genus, the hepacivirus genus, in the flavivirus family. The flavivirus family also includes two other genera: the flaviviruses (e.g., yellow fever virus) and the animal pestiviruses (e.g., bovine viral diarrhea virus (BVDV) and classical swine fever virus (CSFV)). All members of the flavivirus family have enveloped virions that contain a positive-strand RNA genome encoding proteins by translation of a single long open reading frame (ORF).

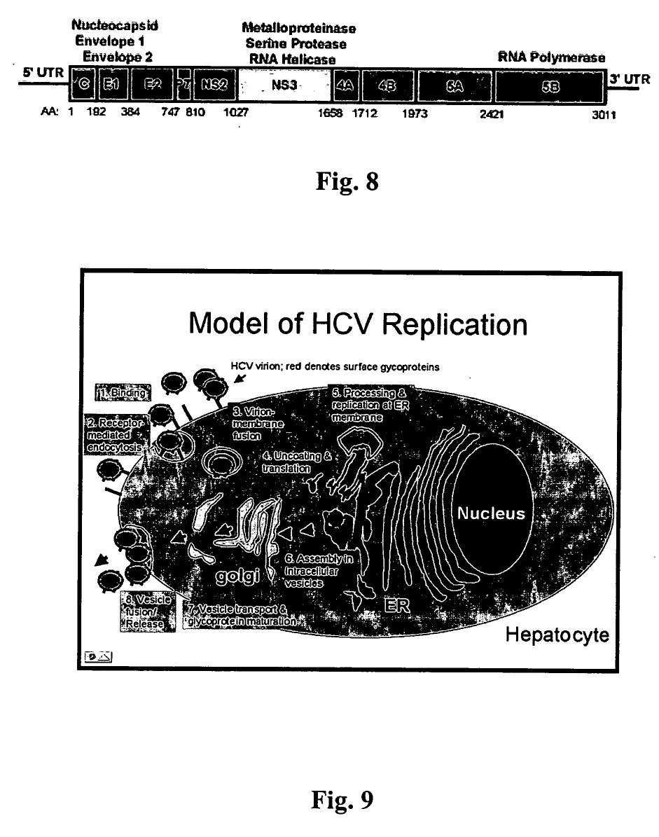

HCV was first identified from non-A non-B infectious plasma in 1989. HCV is an enveloped virus with a single-stranded, linear, positive-sense RNA genome approximately 9.6 kb in length. A highly conserved, 5′-untranslated region of about 340 nucleotides precedes the translation initiation codon. There is also a 3′-untranslated region of variable length (consisting of a short, poorly conserved sequence (28-42 nucleotides), a poly(u)/polypyrimidine tract and a highly conserved 98-base element). (Houghton, M. “Hepatitis C viruses” in: Fields, B. N. et al. eds. Fields Virology 3rd ed. Philadelphia, Lippincott—Raven, 1996:1035-1058.) The RNA genome also carries a single long ORF that encodes a polyprotein of about 3011 amino acids. Translation of the ORF is mediated via an internal ribosome entry site (IRES) that is located in the 5′ non-translated region (NTR) (Tsukiyama-Kohara et al., J. Virol. (1992), 66:1476-1483.) The resulting polyprotein is cleaved co- and post-translationally by concerted action of cellular (host) proteases and two viral proteases into at least 10 different products. (ee FIG. 8.)

The structural proteins, core (C) and the envelope glycoproteins (E1 and E2) are the major constituents of the virus particle. (Kaito et al., J. Gen. Virol., (1994), 75: 1755-1760; Yasui et al., J. Gen. Virol. (1998), 72: 6048-6055; Op De Beek et al., J. Gen. Virol., (2001), 82: 2589-2595.) The glycosylated E1 and E2 molecules are anchored inside the lumen of the endoplasmic reticulum (ER); the C protein remains on the cytosol side. (Houghton, M. “Hepatitis C viruses” in: Fields, B. N. et al., eds. Fields Virology, 3rd ed. Philadelphia, Lippincott—Raven, 1996:1035-1058.)

The HCV E1 and E2 glycoproteins contain a large N-terminal ectodomain, a hydrophobic transmembrane region, and a C-terminal cytoplasmic tail. The ectodomains are located in the ER lumen and the transmembrane domains span the membrane of the ER. The ectodomains of HCV E1 and E2 are modified by N-linked glycosylation. The glycans on E1 and E2 play a major role in the folding of these proteins, partly by interacting with the ER chaperones, including calnexin. The N-termini of E1 and E2 contain a sequence for initiation of translocation into the ER, and when fused to a reporter protein, these sequences function as signal peptides. After translocation of their ectodomain into the lumen of the ER, HCV envelope proteins are cleaved from the polyprotein by a host signal peptidase. After cleavage, E1 and E2 assemble as a noncovalent heterodimer. The transmembrane domains of E1 and E2 play a role in the assembly of the heterodimer.

Accumulation of HCV envelope glycoproteins in the ER or an ER-like compartment may be necessary for the formation of the enveloped particle. ER retention of HCV E1 and E2 has suggested that specific signal(s) present in E1 and E2 are responsible for its subcellular localization to the ER. By using chimeric proteins made between E1 or E2 ectodomains and proteins normally expressed at the cell surface, the chimeric proteins do not accumulate in the ER. This indicates that the transmembrane domains and/or the cytoplasmic tails of E1 and E2 contain ER retention signals.

In one embodiment, the invention provides for the use of chimeric E1 or E2 ectodomain fusion proteins (i.e., “chimeras”) in applications relating to HCV receptor binding and fusion. In another embodiment, the invention provides for the use of full-length E1 and E2 proteins in application relating to HCV receptor binding and fusion. Prior methods focused on using chimeric E1 or E2 ectodomain fusions to avoid ER accumulation. However, the prior methods did not observe syncytium formation when using E1 or E2 ectodomain chimeras in fusion assays. But the present methods provide the unexpected finding that at low cell culture temperatures, native, full-length E1 or E2 proteins can mediate cell membrane fusion and syncytium formation. Further, the present methods provide the unexpected finding that at low cell culture temperatures, full-length E1 and E2 can be transported to a cell surface and mediate cell membrane fusion with observed syncytium formation. Thus, in one embodiment, the invention provides methods relating to fusion that use full-length E1 and E2, where full-length E1 and E2 are present at higher levels on a cell-surface when the cell-culture temperatures are from about 20° C. to about 34° C. as compared to methods with cell-culture temperatures that are at about 37° C. In another embodiment, the invention provides methods relating to fusion that use mutant E1 and E2 comprising at least an ectodomain and a dimerization domain, where the mutant E1 and E2 are present at higher levels on a cell-surface when the cell-culture temperatures are from about 20° C. to about 34° C. as compared to methods with cell-culture temperatures that are at about 37° C.

The HCV non-structural (NS) proteins comprise NS2, NS3, NS4A, NS4B, N5A and N5B. The p7 protein is a viroporin and is probably responsible for the flow of calcium ions from the endoplasmic reticulum into the cytoplasm. (Griffen et al., FEBS Lett. (2003), 535: 34-38.) NS2 and the amino terminal domain of NS3 constitute the NS2-3 proteinase that mediates the cleavage between NS2 and NS3. The NS2-3 proteinase may be a cysteine-proteinase that is activated by interaction with the cellular chaperone Hsp90. (Pallaoro et al., J. Virol., (2001), 75: 9939-9946; Waxman et al. Proc. Natl. Acad. Sci. U.S.A., (2001), 98: 13931-13935.) The amino terminal domain of NS3 carries a serine-type proteinase activity that forms a stable heterodimeric complex with NS4A. (Lin et al., J. Virol., (1994), 68: 8147-8157; Bartenschlager et al., (1994), J. Virol., 68: 5045-5055; Bartenschlager et al., (1995), J. Virol., 69: 7519-7528; Failla et al., (1994), J. Virol., 68:3753-3760.) The carboxy terminal domain of NS3 harbors an ATPase/helicase activity capable to unwind double stranded nucleic acids. NS3 has been shown to be indispensable for infectivity in vivo. (Kolykhalov et al. J. Virol., (2000), 74: 2046-2051.) NS4B is a highly hydrophobic protein that appears to induce distinct membranous vesicles of unknown origin. (Egger et al. J. Virol., (2002), 76:5974-5984; Hugle et al., Virology, (2001), 284:70-81.) NS5A (as well as E2) may interfere with IFN-α antiviral effects, possibly through a direct interaction with the protein kinase PKR. (He and Katze, Viral Immunol., (2002), 15:95-119; Gale et al., Virology, (1997), 230: 217-227.) NS5B is the RNA-dependent RNA polymerase (RdRp) that is able to initiate RNA synthesis. (Behrens et al., 1996; Lohmann et al., 1997, Luo et al., 2000; Zhong et al. 2000; O'Farrell et al. 2003; Sun et al. 2000; Kao et al. 2000.) The NS proteins have been localized to the membrane of the ER, suggesting that it is the site of polyprotein maturation and viral particle assembly. (Houghton, M. “Hepatitis C viruses” in: Fields, B. N. et al., eds. Fields Virology, 3rd ed. Philadelphia, Lippincott—Raven, 1996:1035-1058.)

### HCV Envelope Glycoproteins and Virus Entry

Viral envelope proteins are involved in the early steps of the viral life cycle. The viral envelope proteins at least have two roles in virus entry: (1) in receptor binding, and (2) in inducing fusion between the viral envelope and a cellular membrane. At least two classes of viral fusion proteins have been identified (Jardetzky and Lamb, Nature (2004), 427: 307-308). Based on sequence similarities and structural homologies with other fusion proteins from closely related viruses, HCV envelope glycoproteins E1 and E2 may belong to class II fusion proteins. But contrary to what is observed for other viral envelope proteins of this class, HCV E1 and E2 are highly glycosylated and are not matured by a cellular endoprotease cleavage.

Infection begins with attachment of the virion to the surface of the host cell. Attachment is mediated by the binding of a protein present at the surface of the virion to a molecule on the cell surface, acting as a virus receptor. Additional cell surface molecules, or coreceptors, may also be involved in attachment. The envelope glycoproteins E1 and E2 are the viral components present on the surface of the HCV particle that are most likely responsible for binding to a host cell surface receptor(s) and coreceptor(s). Validation of a viral receptor or co-receptor requires proof that the putative receptor is necessary for infection. Because prior methods for HCV propagation do not allow efficient HCV replication, the identification of receptors has been hampered. However, the present methods allow for efficient and robust HCV replication and production of infectious HCV virions, such that transfection of a non-permissive cell line with cDNA encoding the candidate receptor can be conducted in order to validate that the candidate receptor does confer susceptibility of infection to the non-permissive cell line. Prior cell culture methods for HCV replication did not incubate infected or transfected cells at low temperatures ranging from about 20° C. to about 32° C., or from about 20° C. to about 30° C. Without being bound by theory, the present invention theorizes that the lower temperatures enable more efficient folding of E1 and E2, such that improved HCV assembly of infectious virions is obtained.

### HCV Glycoprotein Mediated Fusion Assay

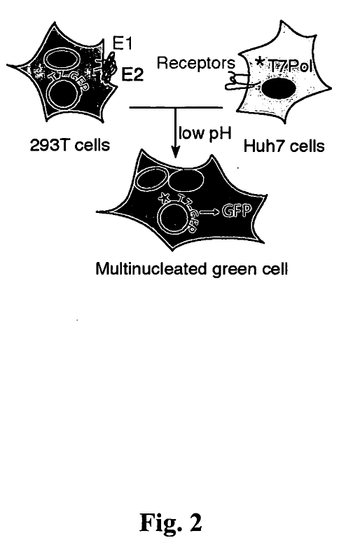

In the basic scheme of the fusion assay of the present invention (see FIG. 2; Example 1; Example 5), there are two types of cells, an “effector cell” and a “target cell.” The “effector cell” can essentially be any cell-type that is easy to transfect and grow. The effector cell comprises (1) a reporter gene under control of an inducible or a non-constitutive promoter, and (2) HCV E1 and E2 proteins expressed on the effector cell surface. In one embodiment, the effector cell is a 293T cell, which contains a GFP reporter gene under control of a T7 promoter and full-length HCV E1 and E2 proteins expressed at the cell surface. The “target cell” can be any cell-type that expresses cell-surface receptors that can specifically bind to E1 and E2 to mediate fusion between the effector and target cells. The target cell can comprise a cell that is derived from normal or transformed liver cells. Also, the target cell comprises a protein or some other agent that can turn-on or activate or otherwise trigger the reporter construct or substance in the effector cell. In one embodiment, the “target cell” is the Huh-7 or Huh-7.5 cell, which expresses on its cell surface receptors that can bind to E1 and E2 and which expresses T7 polymerase intracellularly. When the effector cell and the target cell are incubated together at low pH and low temperature, robust and reliable fusion is observed, either by microscopy (showing syncytium formation) or by detection of GFP fluorescence. In the embodiment depicted in FIG. 2, GFP will only be expressed when the target cell and the effector cell fuse, through the interaction of E1 and E2 with target cell receptors. Because the effector cell does not express T7 polymerase and the target cell does express T7 polymerase, GFP expression only occurs through fusion of the two cell types.

The fusion assays of the invention can rely on or require a low temperature incubation between the target and effector cell. Low temperature conditions for incubation of target and effector cells in fusion assays are generally the same as the low temperature conditions for HCV cell culture methods of the invention. Low temperature conditions for incubation of target and effector cells in fusion assays can comprise, for example, a temperature from about 20° C. to about 34° C., from about 20° C. to about 32° C., from about 25° C. to about 32° C., from about 26° C. to about 32° C., from about 28° C. to about 32° C., from about 25° C. to about 30° C., from about 25° C. to about 29° C., from about 25° C. to about 28° C., from about 25° C. to about 27° C., from about 25° C. to about 26° C., from about 28° C. to about 29° C., or from about 29° C. to about 30° C. In the present invention, in relation to temperature, the term about refers to +/−less than 1° C.

The cell-cell fusion methods of the invention also can rely upon or require a transient exposure of co-cultures of effector and target cells to a low pH condition. If the effector and target cells are capable of fusion because they express HCV glycoproteins and the cellular receptors that mediate their binding and fusion, then the effector and target cell can fuse only if the cells are transiently exposed to low pH. The low pH can comprise, for example, from about pH 4.8 to about pH 6.0, from about pH 5.0 to about pH. 6.0, from about pH 5.0 to about pH 5.5, about pH 5.4, or about pH 5.5. Fusion occurs very inefficiently if the pH is at or greater than 6.2. An exposure to low pH that is less than one second in duration is sufficient for fusion to be enabled. Thus, exposure times to low pH can comprise, for example, from about less than 1 second to about 2 minutes. But in order to have the fusion assays reproducible from well-to-well and experiment-to-experiment, the exposure times to low pH comprise, for example, from about 30 seconds to about 1 minute. Times greater than 2 minutes work, however, cellular damage begins to occur with long incubation times at low pH. Without being bound by theory, the low pH mimics the low-pH environment of the endosome, which HCV viruses normally encounter upon entry into cells and subsequent endocytosis. Without being bound by theory, the low pH appears to be important for the HCV envelope glycoproteins to undergo a conformational change that allows the viral membrane (which is analogous to the membrane of the effector cell or a cell that expresses HCV glycoproteins on its surface) and the endosomal membrane (which is analogous to the target cell membrane or a cell that expresses the requisite cellular receptors to mediate HCV glycoprotein binding and fusion) to fuse, releasing the viral nucleocapsid into the cytoplasm to continue subsequent steps in replication.

Due to their reproducibility and effectiveness, the low-temperature based and/or low-pH based fusion assays of the invention have a wide variety of applications. For example, the fusion assays can be used to identify an HCV receptor. But in this application, the target cell would not comprise a liver cell line that is permissive for fusion. Rather, the target cell comprises a non-liver cell line that expresses CD81 (either endogenously, or by stable or transient transfection). The target cell is also transfected with a cDNA expression construct library that is made from hepatocyte mRNA. Thus, when the target cell containing CD81 and a cDNA library vector is incubated with an effector cell expressing HCV E1 and E2, only those target cells that display both CD81 and another HCV receptor will allow fusion. Upon fusion, the target cell can provide a protein that can specifically activate or promote reporter gene expression, such as a T7 polymerase and a GFP reporter gene under control of a T7 promoter. In this manner, fusion can be easily detected by fluorescence detection, which makes the method amenable to high-throughput application.

Further, as shown at least in Example 5, the E1 and E2 proteins in the fusion assays should at least comprise an ectodomain and a dimerization domain, where the dimerization domain is located in the transmembrane domains of E1 and E2.

In another application, the present fusion assays can be used to identify inhibitors of fusion, where the inhibitors can be peptides, peptidomimetics, small molecules, antibodies, modified peptides, etc. In this application of the fusion assay, the effector and target cells are chosen on the basis that fusion will occur unless the cells are incubated with a compound or substance that will inhibit fusion. Thus, the effector cell can comprise HCV E1 and E2 and a reporter gene, and the target cell can comprise a cell-type known to fuse with HCV E1 and E2, such as Huh-7, and a protein that can activate or promote reporter gene expression. In the basic scheme of FIG. 2, fusion is detected by the presence of GFP expression. Effector and target cells can be incubated in a large number of multi-well plates, such as 96-well plates, to allow for high-throughput screening. In one embodiment, prior to incubation of the effector or target cell, the inhibitor and either the effector cell or the target cell can be incubated first. In this manner, a potential inhibitor can bind to HCV E1 or E2 if it is preincubated with the target cell or a potential inhibitor can bind to a HCV receptor if it is preincubated with the effector cell, thereby potentially preventing fusion.

### Cell Culture Methods for the Propagation and Study of HCV Infection

Studies on the structure and replication of HCV have been limited by the lack of a cell culture system able to support efficient virus replication and produce high titers of infectious virus. Prior methods have tried to develop surrogate models such as the production of virus-like particles or pseudotype viruses, incorporation of HCV envelope glycoproteins into liposomes or development of a cell-based fusion assay. However, these prior methods have encountered difficulties because HCV E1 and E2 have a tendency to misfold (Dubuisson et al., 2000, Curr. Top. Microbiol. Immunol., 242, 135-148), and their folding and assembly is very sensitive to mutations or deletions affecting their transmembrane domains. Therefore, the present methods provide efficient and robust cell culture methods for the propagation of HCV, which therefore provide improved methods for the identification/study of factors and mechanisms that underlie HCV replication in the host cell.

The present methods can be tested to determine whether they provide efficient and robust production of infectious HCV particles by conducting quantitative tests to detect amount (titer) of virus (HCV RNA) that is present in cell-culture supernatants. Quantitative assays for measuring the concentration (titer) of HCV RNA have been developed and are available from commercial laboratories, including a quantitative RT-PCR (Amplicor HCV Monitor™, Roche Molecular Systems, Branchburg, N.J.) and a branched DNA (deoxyribonucleic acid) signal amplification assay (Quantiplex™ HCV RNA Assay [bDNA], Chiron Corp., Emeryville, Calif.). Compared with qualitative RT-PCR assays, these assays are less sensitive with lower limits of detection of 500 viral genome copies per mL for the Amplicor HCV Monitor™ to 200,000 genome equivalents per mL for the Quantiplex™ HCV RNA Assay.

The chimpanzee model can be used to verify whether infectious HCV particles are produced by the methods of the invention. Briefly, chimpanzees that have not been previously exposed to HCV or blood products, and that do not text positive for HCV antibodies and RNA, can be used. HCV particles are injected into the animals, and serum is isolated and assayed for HCV-specific antibodies and for evidence of circulating HCV RNA by RT-PCR. HCV RNA can be detected in serum or plasma within 1-2 weeks after exposure to the virus. Histologic examination of liver biopsy tissues can also be conducted.

In the invention, the “lower” temperature for incubating, growing, propagating or any other step that involves a low temperature cell-culture condition for HCV refers to the air temperature of the compartment or device in which a cell culture is conducted. Low temperature cell culture conditions for HCV can comprise, for example, a temperature from about 20° C. to about 34° C., from about 20° C. to about 32° C., from about 26° C. to about 32° C., from about 25° C. to about 32° C., from about 28° C. to about 32° C., from about 25° C. to about 30° C., from about 25° C. to about 29° C., from about 25° C. to about 28° C., from about 25° C. to about 27° C., or from about 25° C. to about 26° C. In one embodiment, the temperature is at about 28° C. Cells are maintained in tissue-culture incubators, where the shelf temperature or the air temperature of the incubator is set at the low temperature cell culture conditions for HCV of the present invention, as for example, stated in this paragraph.

Generally, cell lines contemplated by the invention can be grown in Dulbecco's Modification of Eagle's Medium (DMEM) or RPMI-1640, or variants thereof, which includes different additions to the media, such as different concentrations of fetal bovine serum, different concentrations of amino acids, different concentrations of antibiotics, etc. One skilled in the art understands that different types of media can be used and tested for, depending upon the cell to be cultured.

One skilled in the art also understands that passaging of cells in culture is contingent upon the particular cell-type. However, cells are routinely passaged, for example, about twice a week at a dilution of 1:3 or 1:4, depending on confluency.

Propagation of Virus Particles with HCV Clinical Isolates

HCV particles from subjects can be isolated from serum or tissue, and thereafter used to initially infect cell lines in culture such that progeny HCV virions can be produced by the present methods.

When HCV particles are isolated from tissue, the particles can be purified from lysates of cells by CsCl gradient centrifugation or other forms of centrifugation (e.g., non-equilibrium centrifugation using a step gradient). Centrifuged particles band in specific fractions, enabling purification. The density of these HCV-immunoreactive fractions are about 1.14 to 1.18 g/cm³ in sucrose equilibrium gradients and 1.14 to 1.16 g/cm³ in CsCl equilibrium gradients.

It will be understood by those skilled in the art that the particles can be purified to substantial purity by other standard techniques such as selective precipitation with substances such as ammonium sulfate, column chromatography, immunopurification and others (see, for example, procedures described by R. Scopes in Protein Purification: Principles and Practice, Springer-Verlag, New York, 1982; and “Guide to Protein Purification”, Meth. Enzymol 182:619-626, 1990).

For isolates from serum or blood, the serum or blood can be used to initially infect cell lines in culture without purification. However, if purification is desired, filter centrifugation can be conducted where virus particles are separated from serum protein due to the pore size of the filter.

In general, virus particles are incubated with cells in plates, flasks or a bioreactor. Initial incubation is conducted at 37° C. to allow for efficient attachment and entry, although lower temperatures may be used at this step. Initial incubation for virus attachment and entry can be conducted, for example, for a period of about 15 minutes to about 1 hour. After initial incubation, the cell culture medium can be changed. Subsequent to initial incubation, the infected cells are incubated at the low temperatures of the invention, from about 20° C. to about 34° C. Cell culture medium can be changed regularly, where with each change, the supernatant can be harvested for purification of virus particles. The purification of supernatants can be conducted by centrifugation and the particles can be tested for infectiousness.

Propagation of Virus Particles Containing the HCV Genome or Portions Thereof

In general, cDNA construct(s) comprising the HCV genome are transfected into a cell line permissive for replication, such as Huh-7. For each transfection well, cell number can be, for example, from about 1×06 to about 1×10⁷, at about 60-70% confluency in plates or flasks. After transfection, the cells are incubated at the low temperatures of the invention, from about 20° C. to about 34° C. Cell culture medium is changed regularly until the supernatant is ready for harvest. The supernatants can be concentrated by ultra-centrifugation in order to concentrate virus particles such that infectious titres can be obtained. Harvested supernatants containing virus particles or titred virus particles can then used to infect permissive cell lines for further propagation.

Prior methods use transfection in order to bypass entry steps, but these methods did not reproducibly yield replication nor assembly and release. In contrast, the low-temperature based methods of the invention are not limited to single-rounds of transfection to produce particles. Because the low-temperature methods of the invention allow for native HCV glycoprotein mediated fusion, entry steps do not need to be bypassed. Further, the low-temperature methods of the invention are suspected to allow reproducible assembly and release.

Because the low-temperature methods of the invention improve HCV glycoprotein function, the low-temperatures can be used in the incubation of cells transfected with plasmids comprising subgenomic replicons of HCV that involve HCV glycoprotein proteins. Further details regarding HCV clones can be obtained in US Publication No. 2002/0102540, which is hereby incorporated by reference.

Pseudotype Particles

Recently, infectious pseudotype particles (HCVpp) have been generated, and they can comprise unmodified HCV envelope glycoproteins displayed onto retroviral cores (Bartosch et al., 2003, J. Exp. Med., 197: 633-642; Hsu et al., 2003, Proc. Natl. Acad. Sci. USA, 100: 7271-7276). Retroviruses can be platforms for HCV pseudotype particle assembly because their cores can incorporate a variety of cellular and viral glycoproteins (Ott, 1997, Rev. Med. Virol., 7: 167-180; Sandrin et al., 2002, Blood, 100:823-832) and they can easily package and integrate genetic markers into host-cell DNA (Negre et al. 2002, Curr. Top. Microbiol. Immunol., 261: 53-74). The subcellular localization of HCV envelope glycoproteins is in the endoplasmic reticulum (ER). ER retention of HCV glucoproteins is leaky, and in overexpression conditions, a small fraction of E1 and E2 can reach the plasma membrane where assembly of HCV pseudotype particles supposedly occurs. But prior methods for the production of HCVpp is inefficient, because there is high tendency of HCV E1 and E2 misfolding when they are expressed at a high level (Dubuisson et al., 2000, Curr. Top. Microbiol. Immunol., 242, 135-148). Thus, the present invention provides methods involving the use of HCVpp, where the propagation of HCVpp is conducted at lower temperatures.

HCV-pp can be produced by transfecting an appropriate cell, such as human 293T cells, with three expression vectors: (1) encoding an E1E2 polyprotein (or alternatively, encoding an E1-E2-p7 polyprotein) under control of an appropriate promoter, such as a CMV promoter, (2) encoding retroviral core proteins (gag-pol) under control of an appropriate promoter, such as a CMV promoter, and (3) a packaging-competent retrovirus-derived genome harboring a reporter or marker gene, such as a GFP gene (the vector will comprise a nucleic acid comprising a retrovirus packaging signal and the marker gene flanked by the retrovirus 5′ and 3′ LTRs) under control of an appropriate promoter, such as a CMV promoter. Other reporter genes can be used, as described herein, including dsRed. After transfection of the cell with the expression vectors, particles are harvested from the supernatant of transfected cells and the particles are purified by ultracentrifugation. The incubation of the cell after transfection can be conducted at lower temperatures, i.e., from about 20° C. to about 34° C., in order to increase the efficiency of HCV-pp production.

The purified particles can then be used in methods that relate to HCV glycoproteins. For example, HCV-pp can be used to identify cellular receptors that mediate attachment and/or fusion of HCV. HCV-pp can be incubated with non-hepatocyte cells that contain a hepatocyte cDNA expression library vector. Successful transduction with pseudotyped particles results in cells producing the fluorescent marker. The fluorescent cells indicate not only that the HCV envelope glycoproteins were able to mediate fusion or transduction of that particle, but subsequent steps of retroviral nucleocapsid disassembly and novel protein synthesis also occurred. Another methods to identify cells expressing candidate receptors can comprise screening for successful replication by RT-PCR. The incubation of the cell with the pseudotyped particles can be conducted at lower temperatures, i.e., from about 20° C. to about 34° C. Further details about the generation of pseudo-particles containing functional HCV E1 and E2 envelope proteins can be obtained in WO 2004/024904, which is hereby incorporated by reference.

### Candidate Receptors for HCV

Several candidate receptors for HCV have been proposed. Because of the physical association of HCV with low- or very-low density lipoproteins (LDL or VLDL) in serum, the LDL receptor has been proposed as a putative candidate receptor for HCV. Although the LDL receptor has been shown to mediate HCV internalization by binding to virion-associated LDL particles, there is no clear evidence that the LDL receptor is a major receptor for HCV pseudotype particles (Bartosch et al. 2003, J. Exp. Med., 197:633-642).

By using a truncated and soluble form of E2, CD81 tetraspanin and the scavenger receptor class B type I (SR-BI, a high density lipoprotein-binding molecule) were proposed as putative receptors for HCV (Pileri et al., 1998; Scarcelli et al., 2002). Both CD81 and SR-BI were shown to be necessary for HCVpp entry. Antibodies directed against CD81 and SR-BI and a soluble domain of CD81 can reduce the infectivity of HCVpp's. Cells that are permissive to HCVpp infection are of liver origin and co-express CD81 and SR-BI. However, there are other cell lines that coexpress CD81 and SR-BI that are not of hepatic (liver) origin.

For example, the Table below lists which cell lines were permissive to HCVpp infection (Bartosch et al., (2003), J. Biol. Chem., 278(43):41624-41630). Only cell lines of hepatic origin that expressed both CD81 and SR-BI were permissive to HCVpp infection.

TABLE 2
Expression of CD81 and SR-BI and Permissiveness to Infection

| | | | | | Permis- |
|---|---|---|---|---|---|
| | | LDLr | CD81 | SR-BI | siveness |
| | | expres- | expres- | expres- | to HCVpp |
| Cell Line | Tissue | sion | sion | sion | infection |
| Huh-7 | Hepatocellular | ++ | + | + | ++ |
| | carcinoma | | | | |
| PLC/PRF/5 | Hepatoma | + | ++ | + | + |
| Hep3B | Hepatocellular | +/− | + | + | + |
| | carcinoma | | | | |
| HepG2- | Hepatocellular | + | ++ | ++ | + |
| CD81 | carcinoma | | | | |
| HepG2 | Hepatocellular | + | − | ++ | +/− |
| | carcinoma | | | | |
| CHO- | Chinese | − | ++ | ++ | − |
| CD81/SR-BI | hamster ovary | | | | |
| CHO | Chinese | − | − | − | − |
| | hamster ovary | | | | |
| HeLa | Cervix | + | + | ++ | − |
| | adenocarcinoma | | | | |

Thus, the data suggests that additional hepatic-cell specific molecule(s) are necessary for HCV entry. Other molecules interacting with HCV E1 and E2 have also been proposed as candidate receptors. They include the mannose binding lectins DC-SIGN and L-SIGN, glycosaminoglycans and the asialoglycoprotein receptor (ASGP-R). However, the potential role of these proteins in HCV entry has not yet been demonstrated.

Validation of a viral receptor or co-receptor requires proof that the putative receptor is necessary for infection. HCV pseudotype particles that contain fully functional E1 and E2 envelope glycoproteins can mimic the function of native HCV particles, and therefore provide a model to study the early steps of the HCV life cycle. Thus, in one embodiment, the invention provides methods for screening candidate receptors by using HCV pseudotype particles, where cells expressing a candidate receptor are cultured with HCV pseudotype particles at low temperatures that range from about 20° C. to about 34° C.

For example, HCV-pp can be incubated with non-hepatocyte cells that contain a hepatocyte cDNA expression library vector. Successful transduction with pseudotyped particles having a reporter gene results in cells producing the fluorescent marker. The fluorescent cells indicate not only that the HCV envelope glycoproteins were able to mediate fusion or transduction of that particle, but subsequent steps of retroviral nucleocapsid disassembly and novel protein synthesis also occurred. Another method to identify cells expressing candidate receptors can comprise screening for successful replication by RT-PCR. The incubation of the cell with the pseudotyped particles can be conducted at lower temperatures, i.e., from about 20° C. to about 34° C.

In one embodiment, a cell-cell fusion assay of the present invention can be used to identify an HCV receptor. In this fusion assay, instead of using a liver cell line that is permissive for fusion as the effector cell, the method uses a non-liver cell line (i.e., non-hepatocyte) that expresses CD81, preferably stably expressing CD81. This cell line is transfected with a cDNA library made from hepatocytes. For example, in the embodiment of the fusion assay where the effector cell contains a GFP reporter and the target cell contains a protein or other substance that turns on the GFP reporter, only target cells (non-liver cell expressing CD81) that contain a library construct encoding a hepatocytic-HCV receptor will allow fusion and therefore become ‘green’ due to GFP expression. GFP expressing cell fusions can be isolated by FACS, which allows for the isolation of the cDNA for the putative HCV receptor (by PCR or RT-PCR, for example).

In one embodiment, screening for candidate receptors is conducted by using native HCV particles. For example, cells expressing a candidate receptor are incubated with native HCV particles (or mutant HCV particles that contain all of the HCV polyprotein products). As stated for pseudotyped particles, the initial incubation of HCV particles with cells can occur at lower temperatures or at normal physiological temperatures, and if the initial incubation occurs at a normal physiological temperature, after a sufficient time allowing for binding (attachment) and entry, the cell culture temperature is then lowered. Cells expressing candidate receptors can be screened for successful replication by RT-PCR or for successful production of new infectious virions by plaque-assay.

Screening for candidate receptors can occur by generating cell lines that stably express a candidate receptor(s) or by transiently transfecting an expression vector(s) encoding the candidate receptor(s) into the cell. Further, screening can also occur by transfecting a cell with a library of expression vectors, where the library comprises a plurality of vectors expressing different products.

### Methods for Identifying Drugs That Inhibit HCV Propagation or Fusion

The invention also includes methods for identifying drugs that can inhibit HCV at any one of the stages of its lifecycle. Thus, the general method for identifying drugs comprises incubating a candidate drug with a permissive cell line (cells that are permissive for HCV propagation can include, for example, PLC/PRF/5, Hep3B and HepG2-CD81) and with HCV particles (or incubating a candidate drug with a permissive cell line and transfecting the cell line with an HCV genome). Drugs that can inhibit HCV attachment, entry, fusion, replication, assembly, virion release, and efficient production of new infectious particles can be tested, because the present invention allows for the efficient production of HCV infectious virions by the culturing infected/transfected cells at lower temperatures. The efficient production of HCV infectious virions inherently shows the successful re-creation of the entire HCV lifecycle in vitro. Thus, after incubation with a candidate drug, if new infectious virions are not produced, or if they are produced at substantially lower titers (as determined by plaque assay), then this indicates that the candidate drug may be successful in inhibiting HCV propagation. The stage at which HCV propagation was inhibited can also be tested for. If the drug inhibited HCV attachment, entry, fusion or replication, then RT-PCR assays should not detect HCV genomes. To determine whether the drug inhibited attachment, fusion or entry, as opposed to replication, further studies can then be conducted using the methods for detecting HCV fusion as described below. If the RT-PCR assays do detect HCV genomes, then this indicates that HCV propagation is blocked in some post-replication stage, which can then be tested for by biochemical means.

The invention also provides methods for identifying drugs that inhibit HCV fusion by using the HCV fusion assays of the invention. For example, an effector cell or a target cell can be preincubated with a candidate drug. The candidate drug can be preincubated with effector cells for testing whether the drug inhibits fusion by binding to HCV E1 and/or E2. The candidate drug can be preincubated with target cells for testing whether the drug inhibits fusion by binding with an HCV cellular receptor, such as CD81. The candidate drug can be added to the co-culture of effector and target cells, before the low-pH step of the assay, for testing whether the drug inhibits the conformational change in HCV E1 and/or E2 that occurs with acid treatment. The effector cell and the target cell are preselected on the basis that they do indeed fuse to each other. Detection of whether the candidate drug prevents fusion can be in at least one of two ways.

First, a reporter gene present in either the effector or target cell can be contingent upon fusion, where the contingency can be provided by placing a reporter gene under control of a promoter, like T7, in either the effector or target cell, and placing the protein that binds to the promoter in the other cell. In this first case, cells that do not express the reporter (and therefore, do not turn green) indicate that the candidate drug might be an inhibitor of HCV E1/E2 mediated fusion. Such a screen can be conducted in a high-throughput manner using multi-well plates, where different candidate drugs are placed in different wells. Either the effector cell or target cell is then added to each well for preincubation with the drug. Then the effector cell or target cell, i.e., whatever cell that was not used for preincubation, is added to the well—this incubation is conducted at low-temperature from about 20° C. to about 34° C.

A second method for detecting whether a drug can inhibit fusion is essentially the opposite for the first method. The second method also uses an effector and target cell combination where fusion is known to occur. However, either the effector or target cell contains a reporter gene construct that is active unless fusion occurs. The target or effector cell that does not contain the active reporter gene construct contains an inhibitor of the reporter gene. In this manner, when fusion occurs, the reporter gene construct will be turned off. Therefore, when fusion is inhibited by a drug, there will no reporter activity. Like the first method, the drug candidate is preincubated with either the effector or target cell. After preincubation, the other cell is added and the mixture is incubated at low-temperature from about 20° C. to about 34° C. This second method can be conducted in a high-throughput manner using multiwell plates.

For both the first and second methods, the reporter gene construct can comprise a GFP gene. Fluorescence from GFP in individual wells can be detected by multiplate fluorescence readers. GFP fluorescence can be quantitated by such readers. This is important because in each individual well, there is a population of effector and target cells, where each cell may not be inhibited to fuse. Therefore, each well that contains a drug candidate should be compared to wells that do not contain a drug candidate. Wells that do not contain a drug candidate can serve as negative controls, or baseline readouts for fluorescence quantitation. In this manner, wells that contain a drug candidate that inhibits fusion and thereby prevent reporter gene expression, i.e., the first method, should present a fluorescence intensity that is less than the negative control. Wells that contain a drug candidate that inhibits fusion and thereby allows reporter gene expression, i.e., the second method, should present a fluorescence intensity that is greater than the negative control.

### Methods for Identifying Mutations that Inhibit HCV Function or Propagation

Comprehensive libraries of mutations can be generated with respect to the whole HCV genome or with respect to particular regions of the genome. In certain embodiments, transposon-based or retrovirus integrase-based mutagenesis can be used to attempt a saturating or near-saturating mutagenesis, such that: (a) each member of the library contains a single insertion mutation in the coding region(s) of interest, and (b) there is close to one independent mutation for every nucleotide of the coding region(s) of interest.

Thus, a genetic approach can be used to identify mutations important for HCV function or propagation. Mutational analysis is a powerful strategy for analyzing the structure and function of genes. Conventional methods of mutant analysis require each viral mutant to be isolated, stored and analyzed separately. This is important because most viral mutants of interest, i.e. those that are replication-defective, cannot be positively selected for as infectious virions. Thus a saturating mutagenesis of a viral sequence of significant length becomes a time- and labor-intensive process. However, genetic footprinting overcomes many of the limitations associated with conventional approaches. It allows a very large set of precisely defined mutations to be made and analyzed en masse in order to define functionally essential regions in a sequence of interest. This technique has been applied to the gag region of Moloney murine leukemia virus (MoMLV) and has allowed the mapping of domains in Gag essential for viral replication.

Thus, comprehensive libraries of mutations can be made in respect to the coding regions for HCV, including E1 and E2. The libraries of mutations can be selected for envelope protein function en masse using two approaches, for example. The first is a cell-cell fusion assay provided by the present invention (see Example 1 and FIG. 2 for a basic scheme), which results in the expression of a fluorescent reporter when cells expressing functional HCV envelope proteins are co-cultivated with liver cells at low-temperatures and exposed to low pH. This mixture of cells can then be sorted using a fluorescent activated cell sorter (FACS) and to thereby select cells expressing mutants that permit fusion. Library DNA isolated from these cells can be analyzed by genetic footprinting to identify domains in the envelope proteins that are involved in fusion.

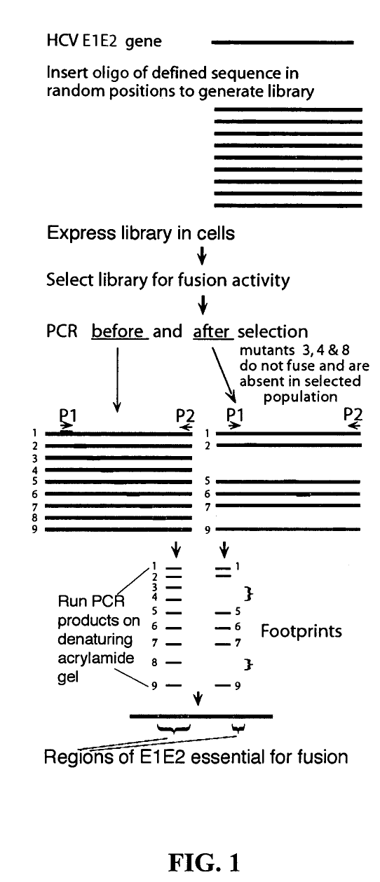

First, a comprehensive library of mutants can be generated, using, for example, either a retroviral integrase or a bacteriophage transposase for the mutagenesis. Each mutant bears a single insertion of a defined oligonucleotide at a random position in the sequence of interest (see FIG. 1). This pool of mutants is subjected en masse to a selection for gene function. DNA is isolated from the library both before and after selection, and the mutants are analyzed by PCR, using one primer specific to a site in the gene, and a second primer corresponding to the insertion. For each mutation, a PCR product of unique length is generated, the length depending on the position of the insert. For the whole library, this gives rise to a ladder of bands on a denaturing polyacrylamide gel, each band representing a molecularly defined mutation. Mutants in which the insertion disrupts a structure required for gene function fail the selection. The corresponding bands are therefore absent from the ladder obtained after selection, giving rise to a ‘footprint’. The footprints represent regions of the gene that are essential for its function. The sequence of the insertion is known, and its position can be inferred precisely from the electrophoretic mobility of the corresponding band. The precise location and sequence of mutations that disrupt gene function can therefore be determined without the isolation and sequencing of many individual mutants.

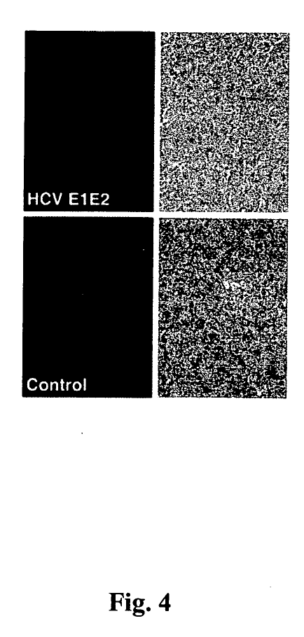

The second method of selection uses retroviral pseudotypes containing HCV envelope proteins to transduce cells and express a fluorescent marker. Viral pseudotypes can be generated using the HCV E1E2 mutants, which are then used to transduce cells. Mutants that permit fusion will result in fluorescent cells (for example, see FIG. 4) that can be sorted from the rest using Fluorescence Activated Cell Sorting (FACS) and the DNA made from such selected cells can be analyzed in parallel using genetic footprinting. Both of these methods can be used to define domains of the envelope proteins that are important for viral binding and fusion.

Individual fusion-defective mutants can also be characterized in detail. Specific envelope protein mutations can be isolated and individually tested for fusion activity. Those that are fusion-defective can be subjected to further studies to determine whether they bind to putative HCV receptors, are transported through the secretory pathway and are correctly processed. This will help identify the precise roles of different regions of envelope proteins, and offer a more detailed, mechanistic understanding of the proteins.

### High Throughput Applications

The present methods can be used in a high-throughput manner because cells can be grown in individual wells of multi-well plates, including microplates. Microplates can comprise polycarbonate material, and have 96-wells, 384 wells, or 1536 wells for example. Individual wells in the 96-well microplate often hold working volumes of about 250 μL. Many companies offer a variety of plate formats and membranes that allow a wide range of functional cell-based assay protocols to be performed. For example, Millipore (Billerica, Mass.) offers a variety of MultiScreen® plates that can be used for both cell culture growth and high-throughput assay testing. The MultiScreen-FL plates are designed for cell viability and fluorescent detection. The plates contain 96 individual wells, where 96 samples can be incubated, washed and assayed. Such plates allow fluorescent signal to be directly detected and quantitated in the plates without sample transfer. Such plates are also compatible with robotics, such as automated liquid handling systems.

With respect to certain embodiments of the present fusion assays, fluorescent reporter activity in microplates can be detected and quantitated by microplate readers. For example, Molecular Devices (Sunnyvale, Calif.) offers the SpectraMax® M5 microplate reader, that is compatible with microplates having 6-384 wells. Detection modalities include absorbance (UV-Vis), fluorescence intensity (FI), fluorescence polarization (FP), time-resolved fluorescence (TRF) and luminescence (Lum), and therefore fluorescence from GFP can be detected and quantitated. With respect to robotics, Molecular Devices offers the SynchroMax ET Plate Handler that provides plate capacity of up to 120 microplates and can be expanded to 320 plates to create an integrated workstation that provides walk-away automation. ELISA or cell-based assays using absorbance, fluorescence and/or luminescence detection modes with dispensing and microplate cell washing can be automated to further increase the throughput and efficiency.

Thus, with respect to certain embodiments of the fusion assays, effector and target cells can be incubated together in individual wells of microplates for high-throughput screening. When the fusion assays are used for identifying drugs that can inhibit fusion, a different drug candidate can be added to each well for high-throughput testing. The drug candidates can be from small-molecule compound libraries or from randomly generated peptide libraries, for example. If the drug candidate can inhibit fusion between a target and effector cell, then fluorescence in the individual well can either decrease or increase with respect to a control well where no drug is added (for example, the control well can be a well adjacent to a test well). The microplate reader data analysis software can quantitate the difference in fluorescence between test and control wells.

In like fashion, high-throughput screening can be conducted to identify new HCV co-receptors. As discussed herein, cells that express CD81 (either endogenously or by transfection, transient or stable) and are known to be incapable of HCV E1 and E2 mediated fusion can be transfected with liver cDNA expression libraries. These target cells can also express a trigger of a reporter construct, such as T7 polymerase. These cells can be incubated in microplate wells with effector cells that express HCV E1 and E2, and contain the reporter construct, such as a GFP gene under control of a T7 promoter. Those target cells that contain a library construct expressing a HCV coreceptor can allow fusion with the effector cells will result in reporter activation, such as GFP expression. Individual wells that contain GFP expression can then be detected by the microplate reader.

High-throughput screening can also be conducted by FACS, as described in Examples 3 and 4. Flow cytometry allows for rapid screening and sorting of individual cells based on fluorescence emission, including GFP emission. High-throughput screening, whether conducted by microplate or by FACS, can be conducted iteratively, where selected drugs, mutations or candidate receptors are repeatedly tested in the same assay to determine whether same results are obtained. In this manner, false positives can be eliminated.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### EXAMPLES

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the examples described below are provided to illustrate the present invention and are not included for the purpose of limiting the invention.

### Example 1

### HCV Fusion Assay and Applications Thereof

The present invention provides a cell-cell fusion assay with two cell-types, where the assay depends on the presence of functional HCV envelope proteins on the surface of one cell-type (“effector cell”) and the presence of putative HCV receptors on the other (“target cell”). The result of fusion is more robust and reliable when the assay is conducted at lower-temperatures. For example, if incubation between an effector cell and the target cell is conducted at 37° C., then there are only background levels of fusion. But if incubation between an effector cell and the target cell is conducted at lower temperatures, for example, at about 28° C. or at about 30° C., then robust and reproducible fusion is observed.

This cell-cell fusion assay can be used to test many of the mutants that have been generated as described in Example 1. In one embodiment, mutants that prevent fusion prevent reporter gene expression, yet the mutants do express HCV envelope glycoproteins on the cell surface. These mutations indicate regions of the envelope proteins essential for membrane fusion. Furthermore, this assay can be adapted to identify the HCV (co)-receptor and also to identify small-molecule inhibitors of fusion.

A luciferase-based assay has also been reported for fusion of chimeric HCV envelope proteins that contain VSV-G transmembrane and cytoplasmic domains, hereon referred to as “HCV-G chimeras” (Takikawa et al., J. of Virology, 2000, Vol. 74, No. 11, p. 5066-5074). However, HCV-G chimeras are unable to provide robust or reproducible fusion using the HCV-G chimeric proteins in the luciferase-based assay. The prior methods use HCV-G chimeras because over 97% of native E1 and E2 proteins are retained in the endoplasmic reticulum via a retention signal in the transmembrane and cytoplasmic domains (under 37° C. conditions). Substitution of these domains by corresponding domains of the VSVG protein allows the chimeric proteins to be efficiently transported to the cell surface.

Although HCV-G chimeric proteins have been confirmed to efficiently reach the cell surface, they were not fusion-competent. It has been recently been pointed out that the dimerization domains of HCV envelope proteins are present in the transmembrane region of E1 and E2. Without being bound by theory, it is believed that the lack of HCV dimerization domains combined with the presence of a transmembrane domain from a protein that is normally a trimer (VSVG) is in part responsible for a lack of robustness of fusion assays that employ chimeric proteins. Although fusion assays that employ chimeric HCV proteins can be optimized using the present methods, fusion assays of the invention that employ full-length HCV E1 and E2 proteins are preferred.

Thus, in this Example, native HCV envelope protein constructs were used instead of the HCV-G chimeric proteins. Native HCV E1E2 constructs, in general, result in a small fraction of the envelope proteins being expressed on the cell surface under 37° C. conditions, but those small amounts of protein appear to be in a conformation that is fusion-competent in the low-temperature based methods of the present invention. Several different native (i.e., full-length) E1E2 constructs were tested (constructs with E1E2 and p7 were also tested) and resulted in reliable amounts of fusion under low-temperature conditions of the invention. This indicates (1) the chimeric nature of the HCV-G proteins, whereby they are missing the HCV transmembrane and cytoplasmic domains, makes them poor fusion candidates, and (2) low-temperature cell-culture conditions improve HCV glycoprotein mediated fusion.

Fusion between the two cell types resulted in a multinucleated cell expressing a fluorescent reporter protein (GFP). The use of GFP allowed the assaying for rare events, because it allows individual cells to be visualized under the microscope or sorted by FACS. In contrast, a luciferase-based assay requires lysis of all cells in the well followed by a flourimetric assay for luciferase, so low frequency events would be lost.

Fusion was tested in a wide variety of liver cell lines, including Huh7, Huh7.5, Hep3B, and HepG2 cells. Results indicated that Huh7 and the related Huh7.5 cells provided the best fusion with native HCV E1E2.

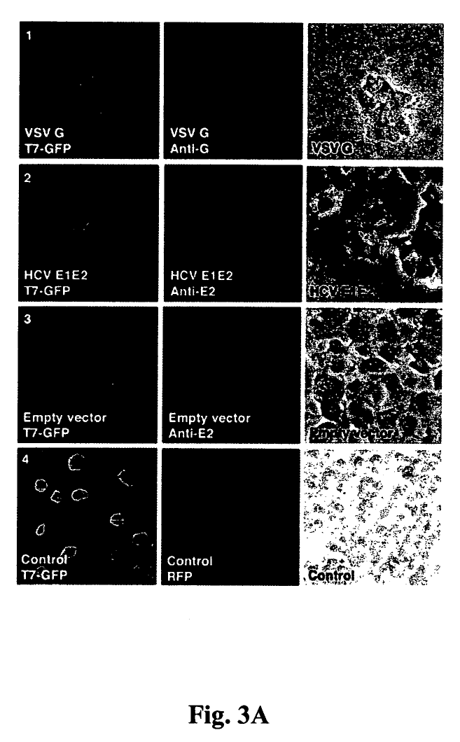

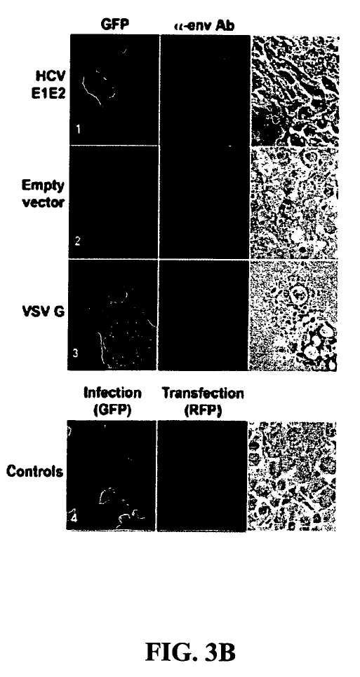

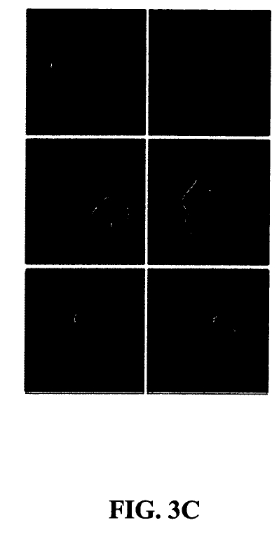

The basic scheme for the fusion assay is shown in FIG. 2 and its application in FIG. 3. Here, 293T cells were used as effector cells to express the HCV envelope proteins. These cells were also co-transfected with DNA that contained the sequence for GFP downstream of a T7 promoter. The target cells, Huh7 liver cells, were made to express high levels of T7 polymerase via the late promoter of the vTF1.1 vaccinia virus (Alexander et al., J. Virol., 66(5):2934-42, 1992). Co-cultivation of effector and target cells allowed HCV envelope proteins on the surface of 293T cells to bind to putative receptors on Huh7 cells. Subsequent transient lowering of the pH to 5.0 mimicked the environment encountered by the virus upon endocytosis (fusion buffers described in (Bonnafous and Stegmann, J. Biol. Chem., 275(9):6160-6, 2000)). The pH can be from about 5.0 to about 6.0 or from about 5.0 to about 5.5, for example, however, pH at about or above 6.0 prevents fusion.

Many members of the two cell-types fused with each other, resulting in multinucleated cells emitting green fluorescence several hours after the fusion. The green fluorescence is due to the expression of the GFP reporter. Cells transfected with empty vector as control showed very few green cells and these were a very faint green (from background levels of expression from the T7 promoter in the absence of T7 polymerase). A second negative control consisted of cells expressing viral envelope proteins that were exposed to an identical buffer, but at pH 7.0; these did not show fluorescence above background. To confirm that fusion was dependent on the presence of HCV envelope proteins on the cell surface, cells were fixed and subjected to immunofluorescence using antibodies to HCV envelope proteins. Each green cell showed high levels of expression of HCV envelope proteins, further evidence that the large, green multinucleated cells were specific to HCV E1E2 mediated fusion. The positive control for fusion consisted of 293T cells expressing VSV-G protein, and these resulted in characteristic large green syncytia, with pH5.0 but not with pH7.0, that stained positive with anti-G antibodies and negative with anti-HCV antibodies. The HCV syncytia were never large, and only consisted of 2-4 cells. This is more akin to the phenomenon seen with some kinds of HIV envelopes, than with VSV-G, which typically resulted in giant syncytia (several hundred cells).

In one embodiment, a general protocol for the fusion assay comprises:

1. Plate effector cells, such as 293T cells, in a suitable tissue culture medium. For example, plate cells in DMEM (Dulbecco's Modified Eagle's Medium)/10% FBS (Fetal Bovine Serum) to a tissue culture plate (such as a 6-well) and incubate 20 hours at 37° C. The cells can be incubated between 18-24 hours, for example, or up to 50 hours with lower cell numbers plated.

Transfect each well of effector cells with a DNA expression construct encoding the full-length E1 and E2 HCV glycoproteins, and an expression construct encoding a reporter, such as GFP under control of a heterologous promoter, such as a T7 promoter or a T7 promoter and an IRES. For the negative control, transfect cells with a vector lacking sequences for the full-length E1 and E2 HCV glycoproteins, but containing all other sequences in the vector, and the expression construct for the GFP reporter. For the positive control, transfect cells with vector containing the sequences for VSV-G protein in place of the sequences for full-length E1 and E2 HCV glycoproteins, and the expression construct for the GFP reporter. Lipofectamine and Plus Reagent in Opti-MEM (all available from Invitrogen Corp., Carlsbad, Calif.) can be used as transfection reagents.

3. Plate human liver target cells known to be able to mediate HCV E1 and E2 fusion (for example, Huh7 or Huh7.5) in DMEM/10% FBS to fibronectin-coated coverslips in a 24-well plate at 8×10⁴ cells per well (range 6×10⁴-1.2×10⁵, more if plated one day prior to infection) or in a 6-well plate at 4×10⁵ cells per well (range 3×10⁵-4.5×10⁵, 5×10⁵-6×10⁵ if plated one day prior to infection), incubate 40 hours at 37° C. (range 18-48 hours).

4. Transfer effector cells to 30° C. incubator (ranges include 20° C.-34° C., 26° C.-32° C., and 28° C.-30° C. for example), and incubate for 22 hours (range 15-30 hours).

5. Infect target cells with 50 μL vaccinia virus vTF1.1 that contains the T7 polymerase gene (Alexander et al. (1992) J. Virol. 66, 2934-2942) in 200 uL serum-free DMEM (or 1 mL in 6-well plate), incubate 3 hours at 37° C. (range 2-5 hours). Also infect one well of 293T cells transfected with T7-IRES GFP for infection control—should give approximately 50% GFP expression rate.

6. Dissociate effector cells using Non-Enzymatic Cell Dissociation Solution following manufacturer's directions (Sigma). Resuspend in DMEM/10% FBS/rifampicin (100 μg/mL)/AraC (10 μM).

7. Remove virus medium from target cells and wash with PBS with calcium and magnesium. Overlay effector cells to 24-well plates at 5×10⁵ cells per well (range 3×10⁵-8×10⁵) or to 6-well plates at 2×10⁶ cells per well. Incubate 5 hours at 37° C. (range 2.5-7 hours).

8. Remove medium and replace with fusion buffer (MES/HEPES pH 5.1 (range pH 5-pH5.5)) for 1 minute, then replace with DMEM/10% FBS/rifampicin (100 μg/mL)/AraC (10 μM), incubate 12 hours at 37° C. (range 5-18 hours).

9. Observe fusion activity using fluorescence microscopy. Also quantitate fluorescence using plate readers.

This basic protocol can be adapted to different applications of the fusion assay of the invention. For example, to identify whether mutations in E1 or E2 affect fusion, the effector cells can be transfected with expression constructs that contain the mutations. To identify whether a drug candidate can inhibit fusion, effector cells transfected with E1E2 constructs can be incubated with the drug candidate prior to incubation with target cells, or alternatively, the drug candidate can be incubated with target cells before the target cells are incubated with effector cells that have been transfected with E1E2 constructs. To identify potential HCV coreceptors, the target cells are cells that express CD81 and are known not to be capable of HCV E1E2 mediated fusion. These target cells are transfected with hepatocyte cDNA libraries, such that transfected cells containing a cDNA encoding a HCV coreceptor will potentially fuse with the effector cell.

Use of the fusion assay for HCV receptor identification. While CD81 has been shown to be necessary for viral entry it is not sufficient. Findings with the SR-B1 and the LDL-R are similar. Thus, there remains a possibility that another molecule, limited to expression in hepatocytes serves as the specific receptor. The cell-cell fusion assay can used to identify such a receptor. For target cells, the cell line should not permit HCV fusion, but should express sufficient amounts of CD81. Identifying a cell line that is not permissive for HCV fusion, yet can be transfected at high efficiencies will not be difficult, since most lines that have been tested do not permit fusion. A non-permissive line is be transfected with a cDNA expression library made from hepatocytes. Cells containing the cDNA(s) for the HCV receptor (and/or coreceptor) will permit fusion and thus turn on the reporter gene (the fused cells will turn green if the reporter gene is GFP). Sorting these green cells and sequencing the specific cDNA(s) provides candidate receptor(s) for HCV. Once the full-length candidate receptor clone has been identified, it is transfected into non-permissive cells. Ability of these cells to now fuse with HCV envelope expressing cells will confirm the role of this protein in fusion. Blocking of fusion by antibodies specific to the candidate receptor, will further verify that the candidate gene is the true receptor.

### Example 2

### Fusion Assay Using Pseudotyped Particles

Pseudotyped particles that contain a retroviral core and HCV envelope glycoproteins ((Hsu et al., Proc. Natl. Acad. Sci. USA, 100(12):7271-6, 2003) (Bartosch et al., J. Exp. Med., 197(5):633-42, 2003) (Flint et al., J. Virol., 78(13):6875-82, 2004) (Lavillette et al., Hepatology, 41(2):265-274, 2005)) have been reported. Transduction with these pseudotyped viruses is dependent on the presence of functional HCV envelope proteins (see FIG. 4). 293T cells were co-transfected with an expression plasmid for HCV E1E2, a plasmid for expressing retroviral Gag and Pol proteins and a plasmid containing the retroviral packaging sequence as well as the fluorescent marker dsRed (plasmid for HCV E1E2 was pcDNA-HE1E2 (Flint et al., J. Virol., 78(13):6875-82, 2004)), (plasmid for Gag and Pol was pCMVDR8.91 (Zufferey et al., Nat. Biotechnol., 15(9):871-5, 1997)), and pCSRW (plasmid for packaging sequence and dsRed (Demaison et al., Hum. Gene Ther., 13(7):803-13, 2002)). Pseudoparticles were harvested, concentrated by ultracentrifugation and used to innoculate Huh-7 cells as described in (Hsu et al., 2003). Cells were incubated for 3-4 days at 37° C. and can be incubated at lower temperatures, such as from about 20° C. to about 34° C., in order to increase production of pseudoparticles.

Purified pseudoparticles were incubated with cells to test transduction. Transduction efficiency was determined by observing red fluorescence from dsRed expression using a rhodamine filter. The highest inoculum transduced was about 50% of cells (FIG. 4, top panel). Cells can be incubated with HCV E1E2 containing pseudoparticles at lower temperatures, such as from about 20° C. to about 34° C. in order to increase transduction efficiency. Control transduction with empty vector instead of pcDNA E1E2, or the chimeric HCV E1-G and E2-G resulted in rare and faint red cells (FIG. 4, bottom panel).

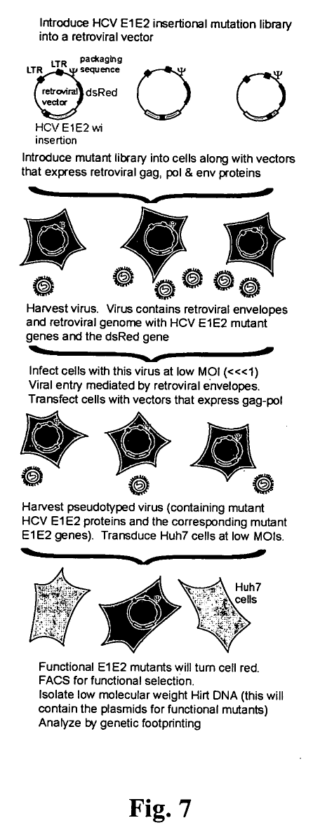

This assay can be used to study mutants in HCV-E1 and E2. Testing can be performed both individually, where one mutant is tested at a time for ability to transduce, and as a pool, where a library of mutants are used to transduce at low MOIs. This method is described in further detail below and in FIG. 7.

### Example 3

### Generation of Large and Extensive Libraries of Mutations is Possible in HCV E1 and E2

Transposon-derived libraries of mutations were made in the chimeric HCV E1-G and HCV E2-G constructs in order to optimize their fusion potential. Chimeric E1-G protein contained the ectodomain of HCV E1 and the signal sequence, transmembrane domain and cytoplasmic tail of the VSV G protein (Takikawa et al., 2000) and chimeric E2 protein, (same as chimeric E1-G, but with E2). Although fusion with these chimeric constructs were not robust enough to be practical for use in the study of mutants, useful information was obtained regarding: (a) size of transposon-derived libraries, and (b) folding of mutant proteins with insertions of five amino acids at random positions along the protein. Specifically, data was obtained that indicates that (a) transposon-derived libraries can be used to generate comprehensive libraries of mutations for any particular HCV protein, including full-length HCV E1 and E2 using the native HCV envelope protein constructs, and (b) a comprehensive mutagenesis of the HCV envelope proteins is unlikely to result in a set of mutants that are mostly folding impaired, and that it will be possible to select HCV mutants for specific functions, including envelope functions, such as receptor binding or membrane fusion, where mutations are made in native full-length HCV sequences as opposed to chimeric sequences containing only portions of HCV glycoprotein coding sequences.

Size of Transposon-Derived Libraries.

The TnsABC transposase (New England Biolabs, Beverly, Mass.) system was used to generate a large and diverse library of mutations in the chimeric HCV E1-G and HCV E2-G proteins. Because these chimeras do not provide efficient fusion in a cell-cell system, a library of mutants in these chimeric proteins is of interest as a proof of concept that large, diverse libraries of mutants can be generated in the HCV envelope glycoproteins. Further, although these chimeras do not provide sufficient fusion, the mutants can be used for other purposes, such as to map the CD81 binding site on E2. The E1 library consisted of 1.5×10⁶ independent mutations, i.e., an average of 285 independent insertions per base of the plasmid. The E2 library consisted of 3.8×10⁵ independent mutations, or ˜63 insertions per base. Since transposases are not completely random in their insertions, the number of mutants required is significantly larger than the number of nucleotides in the mutagenized sequence. However, the size of these libraries is large enough for a saturating or near-saturating mutagenesis. 83 mutants from the two libraries were sequenced. Over 95% of mutants had the expected insertion of 15 bp (base pairs), coding for a 5 amino acid-insertion in 2 out of 3 reading frames, and a stop codon in the third frame. This is important because fidelity of insertion size is necessary both to generate in-frame mutations and to be able to map the exact location (to the nucleotide) of the insertion from the size of its PCR product resulting from the insertion. The results indicate that large libraries of mutations can be reliably and reproducibly generated using a variety of integrase and transposase proteins. Therefore, integrase and transposase proteins can be used to generate comprehensive libraries of mutations for any particular HCV protein, including full-length HCV E1 and E2 using the native HCV envelope protein constructs.

Folding of Proteins with Insertional Mutations.

Mutant proteins generated by using transposases, as described above, contain an insertion of five amino acids. A concern is that such insertions might not only disrupt fusion but might disrupt protein folding. If folding was disrupted for a large fraction of the mutants, it may be difficult to determine the roles of those corresponding regions in fusion. This is because in order to be successfully used in a cell-cell fusion assay, at least a fraction of the mutant envelope protein needs to be correctly folded and transported to the cell surface. Folding of HCV envelope glycoproteins was a special concern because membrane proteins are known to follow more stringent rules for protein folding and for transport out of the endoplasmic reticulum to the cell surface than soluble proteins. Furthermore, another concern was that the insertion might result in an unpaired cysteine in one of the three reading frames (note, the nucleotide sequence is the same for the insertions, but the amino acid sequence varies with reading frame). For mutations that are situated in the extracellular domain of a membrane protein, the unpaired cysteine would be in an oxidizing environment and thus likely to result in a misfolded protein. Approximately a third of the mutants would have an unpaired cysteine. Such an unpaired cysteine did not appear to affect function of Moloney murine leukemia virus envelope protein, which was mutagenized using a different transposase, but the insertion still contained an unpaired cysteine in one of the three reading frames. In order to determine how an unpaired cysteine would behave in HCV envelope proteins, testing for correct folding was conducted as described below.

Transport of a glycoprotein to the cell surface is a well-established and stringent test for correct folding and intracellular transport of that glycoprotein. Misfolded glycoproteins are typically retained in the ER and subsequently degraded, due to a stringent quality control imposed by the ER on proteins expressed on the cell surface. Such a mechanism might have originated so as to not express epitopes that were foreign to the immune system on the surface of cells.

As a surrogate assay for correct folding of membrane proteins, transport to the cell-surface is known to be a more stringent assay than testing reactivity with a limited number of conformation-specific antibodies. Therefore, testing was conducted to determine if mutants from the above-described HCV chimeric protein libraries were expressed on the cell surface. Note that the wild-type or unmutagenized versions of both E1-G and E2-G chimeras are efficiently transported to the cell surface. 27 mutants were randomly selected from the library of mutations in HCV E2-G chimeric protein and 17 mutants from the library of HCV E1-G mutants. These mutants were individually expressed in 293T cells and it was determined if the proteins arrived at the cell surface by immunofluorescence, using anti-HCV antibodies in non-permeabilized and permeabilized cells, and BDI198 mouse mAb to HCV-E1 (Biodesign International). Presence of surface staining pattern in non-permeabilized cells indicated that the protein was expressed on the cell surface. Staining of permeabilized cells was performed to distinguish between misfolded protein that was retained in the ER and unstable protein that was degraded.

The results from all HCV E2-G mutants analyzed this way are summarized below. Of the 27 HCV E2-G mutants, 7 had stop codons in their insertions (this is expected for 1 out of every 3 mutants). When these stop codons were close to the C-terminus, an almost full length chimera protein was generated that was either efficiently transported to the cell surface or resulted in a soluble (not membrane-anchored) protein as seen by a Golgi pattern of staining that is typical for soluble secretory proteins. When the stop codons were further away from the C-terminus, the resulting truncated proteins appeared to be rapidly degraded because cells expressing these mutants did not show any staining. Of the remaining 20 with read-through insertions, 15 (75%) were efficiently transported to the cell surface. Similarly for HCV E1-G mutants, 6 out of 17 had stop codons in their insertions, and each of those depending on how close the stop codon was to the N-terminus, was either degraded or retained in the ER (FIG. 4 shows a representative set of E1-G mutants). Of the remaining 11, 9 (82%) were efficiently transported to the cell surface. The presence of the unpaired cysteine in the insertion did not impair transport. Thus for both E1-G and E2-G chimera mutants, most mutants (75% or greater) appear to fold correctly.

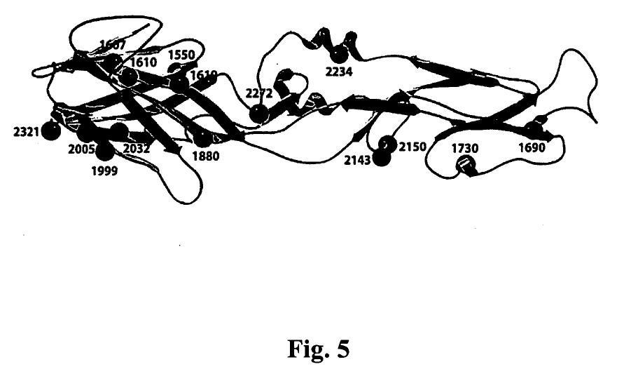

The location of the mutations in E2-G were mapped on to the predicted structure of HCV E2 protein (see FIG. 5). Not all mutants could be mapped, because the model does not cover the entire E2 sequence. Blue spheres indicate mutants that were transported to the cell surface and red spheres indicate those that were either degraded or retained in the ER. It was not obvious if the secondary structure of the region around the mutation played any role in its being correctly folded. For example, mutants that appeared to fold correctly were not preferentially located in regions predicted to tolerate insertions, such as large loops.

The large number (over 75%) of correctly folded mutants might be surprising to some but is not entirely unexpected. Given the size limits on viral genomes, one might intuitively expect viral proteins to have evolved to maximal efficiency, such that every little domain became essential, and thus intolerant of further change. However, evidence with HCV and retroviral sequences seems to point to the contrary, as does a comprehensive mutational analysis of the Moloney murine leukemia virus envelope protein. It can be speculated that RNA viruses might have evolved especially robust protein domains so as to withstand the more frequent rate of mutation that occurs with RNA-dependent polymerases. Regardless of the origin, what is important is evidence that a comprehensive mutagenesis of the HCV envelope proteins is unlikely to result in a set of mutants that are mostly folding impaired, and that it is possible to select HCV mutants for specific functions, including envelope functions, such as receptor binding or membrane fusion, where mutations are made in native HCV sequences as opposed to chimeras.

### Example 4

### Construction of a Library of Mutations in Full-Length HCV E1E2 Proteins and Selection of these Mutants for Function

Because fusion is not reliable with HCV chimeric proteins, several native E1E2 constructs were used in the fusion assay described in Example 1. These constructs included an (a) expression construct comprising native E1 and E2, (b) an expression construct comprising 30 amino acids of the core, E1, E2 and 35 amino acids of p7 from HCV genotype 1a, and (c) an expression construct comprising the entire core, E1, E2 and p7 from HCV genotype 1b.

“pHE1E2” is an exemplary construct that showed fusion activity and comprised native full-length E1 and E2 (no core or p7). This construct was used for mutagenesis. When contained in its parent vector, the length of the entire construct is ˜8 kb nucleotides in length, which is well within the limits suggested by the makers of the transposase (less than 10-12 kb). Prior to mutagenesis, two acts were conducted: Removing two preexisting PmeI restriction endonuclease sites and verifying that the library of mutations, once made, can be moved into the retroviral vector for the pseudotype fusion assay. This was done for practical reasons, i.e., that in making one mutant library with pHE1E2 that can be used in fusion assays as described in Example 1, this primary library could then be used as a basis for making a secondary library in a retroviral vector that can be used in fusion assays as described in Example 2.

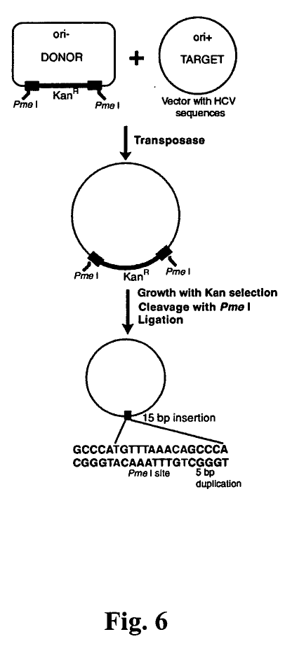

The rationale for removal of the PmeI sites is as follows (also see FIG. 6). Mutagenesis with TnsABC transposase results in the random insertion of a transposable element which is flanked by restriction sites for Pme I. Cleavage with Pme I results in removal of most of the transposable element. It also generates cohesive ends, which can be ligated to create 15-bp insertions, 8-bp of which consist of the Pme I restriction site. The presence of additional, preexisting PmeI sites in the plasmid would not permit these steps. The preexisting PmeI sites were removed by site-directed mutagenesis, using the Stratagene Quikchange site-directed mutagenesis kit. Substitution of two nucleotides in each PmeI site removed the cognate recognition sequence for PmeI, yet maintained the original amino acid sequence of the protein. This new construct designated as pH77E1E2A2Pme was used for generating TnsABC transposase-derived insertional libraries.

There must be restriction endonuclease sites flanking the E1E2 region in pHE1E2 in order to transfer the E1E2 region from pHE1E2 to the retroviral vector for making psuedotyped particles ((“pCSRW” containing the long terminal repeats (LTRs), viral packaging sequence Y, and a chromophore marker for infection (dsRed) (Demaison et al., Hum. Gene Ther., 13(7):803-13, 2002). Flanking endonuclease sites are useful because once the mutation library is made in pHE1E2—the primary library—it can be transferred to the retroviral vector (the secondary library), instead of re-making the library in the retroviral construct. Since an entire library of mutations is to be moved from one vector to another, and not simply a single clone, this process can be designed to be efficient. Inefficient ligation or bacterial transformation or inadequate plating will all result in a loss of diversity of the library. As long as the number of secondary transformants is about 10 to 20-fold higher than the primary library, a loss in diversity is not observed. Loss of diversity can be checked by doing a genetic footprinting reaction on the unselected primary and secondary library. If the pattern and intensity of the bands is the same in both, there is no loss in diversity. Alternatively, if this turns out to be a problem, the E1E2 region can be placed in the retroviral vector first followed by transposon-mediated mutagenesis. The retroviral vector along with the E1E2 region will still be within the size limits dictated by the transposon-mediated mutagenesis.

Generating a Library of Insertional Mutations.

Transposon-mediated mutagenesis involves the integration of ‘donor’ DNA, containing sequences from the ends of the viral/phage DNA, into a plasmid containing the target gene or coding sequence, such as sequences encoding HCV proteins. The insertion of donor sequences into naked target DNA is largely independent of target DNA sequence, giving rise to a near-random set of mutations in the target gene.

Different enzymes can be used to perform the mutagenesis: retroviral integrase, MuA transposase, and a commercially available variant of Tn7 transposase, TnsABC transposase. Since transposases are not completely random in their insertions, the number of mutants required is larger than the number of nucleotides in the mutagenized sequence. The larger the size of the libraries, the more likely it is to result in a saturating library. The method diagrammed in FIG. 7 depicts the generation of an extensive library of mutations in pHE1E2. Some key steps are detailed below.

The libraries can contain a sufficiently large number of clones so as to result in a saturating or near-saturating mutagenesis, i.e., averaging a few hundred integration events per nucleotide of plasmid sequence. This is important because transposition is not entirely a random event: certain sequences of naked DNA are consistently preferred over others. If greater than 1.6×10⁶ primary transformants are obtained, then the library should be large and diverse.

The insertions consist of 15 bp, 10 of which consist of the PmeI restriction site and the remaining 5 bp result from a staggered cut in the DNA placed by the transposase, followed by duplication of the intervening sequence. This duplicated sequence is unique to that insertion site, leaving only 10 bp of nucleotide sequence the same in all insertions.

Even though a major part of the nucleotide sequence is the same, the amino acid sequence varies with the reading frame. In two of the three reading frames it is X-Cys-Leu-Asn-X, or X-Phe-Lys-Aln-X. The identity of amino acid X depends on the flanking sequence. The third reading frame contains a TAA stop codon (X-Val-STOP), resulting in a third of the mutations being truncations.

Approximately a hundred randomly selected mutants are sequenced from the library to verify that insertions have the predicted size and sequence, i.e. they contain an invariant 10 bp sequence flanked by a 5 bp repeat. Typically, 5-10% of mutants in any library have frame-shift mutations due to non-canonical cleavage by the transposase, resulting in either a 4 bp or a 6 bp repeat flanking the insertion. Such frameshift mutations typically result in loss of function, and do not affect the analysis. Note that such non-canonical insertions represent a tiny fraction of mutations at any given nucleotide—the bulk of the signal seen in any band of genetic footprinting is from the 95% insertions that have the canonical insertion.

Selecting Mutants for Function.

Two fusion assays: (1) cell-cell and (2) virus-cell (using pseudotyped particles) can be used to select for HCV E1 and E2 mutants.

Cell-cell fusion assay. Briefly, in this assay, functional HCV envelope proteins are expressed in one cell-type, and the other cell-type consists of cell line that has cellular receptors that can specifically bind to HCV glycoproteins (either by endogenous expression of cellular receptors, heterologous expression or a combination thereof), such as the Huh7 liver cell line. When the cells are co-cultivated at low temperatures and transiently shifted to a low pH, such as pH 5, they fuse. Expression of GFP serves as a reporter for fusion, and this assay was described in detail in Example 1.

This assay can be performed in two stages. First, when the library is made, some mutants are randomly selected, and tested in the cell-cell fusion assay. Since there is no precedent for any structure-function studies of these proteins before, even a modest number of mutants (˜30) when tested individually will significantly enhance knowledge about E1 and E2. Second, the assay is performed on the entire library of mutations, and reporter positive cells are be selected for, such as by FACS.

Virus-cell fusion assay. The second method of selection involves the creation of viral pseudotypes, containing mutant HCV envelope proteins and retroviral cores. Transduction with these pseudotyped viruses is dependent on the presence of functional HCV envelope proteins. FIG. 8 shows the scheme for the selection process. In brief, the library of E1E2 mutants is moved en masse into a pared-down genome of retrovirus (see Example 3). This genome is packaged into virions and delivered via HIV envelope proteins to cells containing HIV receptors, at a multiplicity of infection of <<1. The low multiplicity is important to prevent complementation of mutants. Virions that are subsequently produced contain both the gene and the proteins for a single mutation in E1E2. These pseudotyped virions are used to transduce liver cells. The use of dsRed expression as a marker for successful viral entry at the final stage allows one to use FACS to sort red cells from the mixture. Low molecular weight Hirt DNA (which is rich in retroviral DNA) are prepared from these sorted cells and analyzed by genetic footprinting.

Sorting functional mutants en masse. The fusion assays allow selection of fusion-competent mutations from the library and thus map the domains required for fusion by genetic footprinting. Cells that fluoresce green in the cell-cell fusion assay, and those that fluoresce red in the pseudotype particle assay can be sorted from the mixture using FACS.

For the cell-to-cell assays, selection is performed at different pHs. Mutants fusing at a pH higher than the wild-type have been classic tools to dissect the structure-function relationship of fusion proteins (reviewed in (Hernandez et al., Annu. Rev. Cell Dev. Biol., 12:627-661, 1996)). Thus a footprint that specifically appears at a different pH than that of wild-type protein fusion, may well map domains essential for fusion.

FACS. The argon laser is set at an excitation wavelength of 488 nm and emission wavelength of 530 nm (FACSCalibur, Becton-Dickinson Instruments). Low molecular weight Hirt DNA prepared from sorted cells are analyzed by genetic footprinting. Hirt DNA from as few as 2000 cells yields enough template DNA for an adequate signal in a footprinting reaction.

Genetic footprinting. Cells expressing the E1E2 mutants are selected for envelope protein function using FACS. Low molecular weight DNA is made from these cells (Hirt 1967), which contains DNA from plasmids (or retroviral vectors) that contained functional mutants. This DNA is used for footprinting analysis (diagrammed in FIG. 1). The method of analysis depends on the length of the inserts. The insertions in E1E2 will only be 15 bp in length, with only 10 bp of those being invariant. The other 5 bp arise from target sequence duplication, and thus vary with the location of the insertion. Since 10 nucleotides may be too short for a specific priming site, an alternative strategy for footprinting these inserts is used (Singh, Crowley et al. 1997).

In brief, using Hirt DNA from cells selected for fusion, a 200-300 bp region of E1E2 sequence is amplified. One of the primers is radiolabeled. Since each of the mutants contains a unique PmeI restriction endonuclease site in the insertion, digestion of the PCR products with PmeI produces a ‘ladder’ of bands when run on a denaturing acrylamide gel. These bands are analogous to those generated with the PCR method of footprinting. Footprints indicate which areas of the sequence are essential for fusion. Using multiple primer pairs that each amplifies a 200-400 bp region of the sequence, information is obtained from the entire envelope protein region.

Advantages of genetic footprinting. Several advantages of genetic footprinting over conventional methods of mutagenesis and mutant analysis make it the method of choice for the large-scale mutant analysis of HCV envelope proteins where little is known in terms of structure-function analysis. The characteristics of genetic footprinting that make it both powerful and practical are elaborated below:

1. A large set of molecularly defined mutations can be made and analyzed simultaneously, allowing techniques of saturation mutagenesis to be applied to large stretches of sequence.

2. It does not require mutants to be recovered in order to recognize their effect on gene function. The presence of such mutants in the initial pool, and their failure to be recovered after selection, can be inferred from the footprint. Thus, phenotypes of nonviable mutants can be determined. This is of special significance for viruses, because most mutants of interest, i.e., those that are replication-defective, cannot be positively selected for or recovered as infectious virions.

3. A band corresponding to a particular mutation need not be completely absent in order to recognize the mutant phenotype. Comparing the relative intensity of the PCR products from the ‘selected’ and ‘unselected’ fractions allows recognition of subtle quantitative or temporal defects of a mutation.

4. It provides a means for identifying every site in the sequence of interest that can tolerate an insertion. This might be particularly useful to determine the effects of an inserted epitope tag, which may be used for isolating (e.g., 6-His tag) or localizing (e.g., FLAG tag) replication intermediates.

5. Genetic footprinting also allows considerable variation in the sequence of the mutation. In mutagenizing genes that code for proteins, the insertion or deletion should generally be a multiple of three bases, in order to maintain reading frame. Almost any sequence, except a sequence that is already present in the gene can be introduced as a mutation. For example, the sequence could (in one of the reading frames) encode a peptide that tends to destabilize a particular secondary structure. It could code for amino acids with bulky side chains that might disrupt closely packed domains in a protein's hydrophobic core. It could be a (His)ₙ sequence to aid protein purification, or an epitope tag to facilitate subcellular localization or purification of the protein. It could incorporate a peptide that confers a new functionality to the protein, for example, the ability to be phosphorylated by a specific protein kinase, cut by a specific protease, glycosylated, or transported to a specific cellular compartment. It might be useful to conduct several footprinting analyses of the same gene, each using a different sequence for the inserted oligonucleotide, since one sequence might be tolerated at a particular location while a second sequence with different properties might not.

6. Genetic footprinting allows considerable variation in the size of the insertion. Libraries that contain 36-nucleotide (=12 amino acid) or 15-nucleotide (=5 amino acid) insertions have been generated before. The minimum practical size for the inserted sequence depends on the method chosen for mutant analysis. A PCR-based method requires that the insert be long enough to serve as a specific PCR primer, i.e. a minimum of at least 15 nucleotides, preferably longer. The insert could be as short as 4-8 bases if it were to include a restriction endonuclease site, allowing the analysis described above.

7. Large insertions can effectively map small essential domains. One might intuitively think that in order to map small domains, one needs small mutations, perhaps even point mutations. However, relatively large insertions (36 nucleotides) can effectively map small domains such as the 4 amino acid late domain of retroviral capsid assembly, or the few base pair long bacterial promoter sequences.

8. Finally, because this is a method that examines a sequence exhaustively, it is possible to find essential domains, where one wouldn't intuitively look.

Characterizing Individual Mutants in Detail.

Fusion assay. Each mutant can be tested using the fusion assays described above. Mutations in fusion peptides are often associated with a change in the pH threshold of fusion. Thus, the change in threshold can be tested by performing the fusion assay at higher pH, such as from 5.0 to 6.3.

Those that fail to fuse could be defective in fusion, or a step prior to fusion, such as protein folding, dimerization, transport through the secretory pathway and proteolytic processing.

Binding to receptor(s). The mutant proteins can also be tested for binding to CD81 receptors. Soluble GST-CD81 protein is expressed in bacteria and purified using the GST tag in the protein. The CD81-GST-tagged soluble protein has been tested for binding to HepG2 cells that express HCV E1E2, and performing immunofluorescence with anti-GST antibodies to look for binding of E2 to CD81. HepG2 cells were chosen because they express very low (if any) levels of CD81.

Arrival at the cell surface. Mutants are tested individually for their ability to get transported to the cell surface. Testing for HCV E1E2 proteins on the surface can be conducted by flow-cytometry, e.g. (Dumonceaux et al., J. Virol., 77(24):13418-24, 2003). Antibodies against E1 and E2 can also be used in immunofluorescence studies.

Multimerization. HCV E1E2 proteins are thought to form heterodimers at neutral pH and homotrimers at acid pH. Heterodimerization status can be tested for by using a combination of co-immunoprecipitation and western blotting, as in (Dumonceaux et al., 2003). Cells are lysed in a non-ionic detergent, the post-nuclear lysates immunoprecipitated with anti-E2 antibody, run on SDS-PAGE gels and blotted with an anti-E1 antibody. The presence of E1 on the blots indicates heterodimerization. These mutants can be further tested by subjecting mutant proteins to velocity sedimentation in sucrose gradients. Multimerization defects may point to regions involved in forming contacts between monomers.

### Example 5

### Functional Analysis of Hepatitis C Virus Envelope Proteins, Using a Cell-Cell Fusion Assay

HCV envelope proteins mediate the entry of virus into cells by binding to cellular receptors, resulting in fusion of the viral membrane with the host cell membrane and permitting the viral genome to enter the cytoplasm. In this Example, the invention provides evidence of the development of a robust and reproducible cell-cell fusion assay using envelope proteins from commonly occurring genotypes of HCV. The assay scored HCV envelope protein-mediated fusion by the production of fluorescent green syncytia and allowed the elucidation of many aspects of HCV fusion, including the pH of fusion, cell types that permit viral entry, and the conformation of envelope proteins essential for fusion. It has been determined that fusion could be specifically inhibited by anti-HCV antibodies and by at least one peptide. A number of insertional mutations in the envelope proteins were also generated, and nine of these mutants were tested using the fusion assay. This Example demonstrates that this fusion assay is a powerful tool for understanding the mechanism of HCV mediated fusion, elucidating mutant function, and testing of antiviral agents. The HCV fusion assay described in this Example uses GFP as a reporter, which permits one to measure fusion even when not all cells in the culture form syncytia. The experiments in this Example expand upon those listed in the prior Examples.

Materials and Methods

Cells, Expression constructs and Virus:

293T cells were used as effector cells for the fusion assay. Target cells were Huh-7.5, Huh-7, Hep3B, HepG2/hCD81, HepG2, and FLC4 cells. HepG2 and HepG2/hCD81 were propagated on plates coated with collagen type 1 (˜56 ug/ml in 0.02M acetic acid; BD Biosciences #354236). All cells were cultured in Dulbecco's Modified Eagle's Medium (DMEM) containing 10% Fetal Bovine Serum (FBS), 2.2 mM L-glutamine, 100 units/ml penicillin and 100 μg/ml streptomycin, at 37° C. in a mixture of 95% air and 5% CO2. All cells were regularly tested for mycoplasma (Stratagene #302008) and kept mycoplasma-free.

Constructs expressing envelope proteins were as follows. pcDNA3.1-HE1E2 expressed 22 amino acids from the C terminus of C protein, all sequences from the E1 and E2 proteins of HCV genotype 1a(H) (Flint, M., et al. (1999) J Virol 73:6782-90). Chimeric constructs pCAV340V and pCAV711V consisting of ectodomains of E1 and E2 sequences from HCV genotype 1b, and signal sequence, transmembrane domain and cytoplasmic tail from VSVG; and pCAGVSV to express the VSV-G protein were as described (Takikawa, S. et al., J Virol 74:5066-74). pcDNA3.1 (Invitrogen Corp., Carlsbad, Calif.) was used as empty vector control. pT7IRES-GFP was constructed by cloning EMCV IRES from pNCA-IRES into XbaI/NheI site of pQBI T7-GFP (Quantum Biotechnologies).

Replication defective lentiviral vector pCMVDR8.91 (Zufferey, R. et al., (1997) Nat Biotechnol 15:871-5) was used to express Gag and Pol proteins, and the packaging vector pCSRW (Demaison, C. et al., (2002), Hum Gene Ther 13:803-13) encoded for fluorescent reporter DsRed. To generate pseudotyped particles, the above lentiviral vectors were introduced into cells with expression constructs for either wild type or mutant E1 and E2 proteins as described below.

Vaccinia virus, VTF1.1 (Alexander, W. A. et al., (1992) J Virol 66:2934-42.) was used to express T7 polymerase via the viral late promoter in target cells used for fusion.

Fusion Assay:

T cells, 8×10⁵ cells/per well seeded in 6-well tissue culture dishes 24 h prior to transfection, were transfected with pcDNA3.1-HE1E2, an expression construct for HCV E1 and E2 glycoproteins. For the negative control, cells were transfected with pcDNA3.1, which lacked sequences for the E1 and E2 proteins. For the positive control, cells were transfected with pCAGVSV, an expression construct for VSV-G protein. All cells were co-transfected with pT7IRES-GFP, encoding for GFP under control of the T7 promoter. Lipofectamine and Plus Reagent in Opti-MEM (from Invitrogen Corp., Carlsbad, Calif.) were used for transfection, following manufacturer's directions. Transfections were deemed successful when a control well transfected with a plasmid expressing a fluorescent reporter indicated at least 80% rate of transfection.

To test if in this fusion assay, lower temperatures might result in better folding of the E1 and E2 proteins and more efficient transport to the cell surface, cells were grown at different temperatures between 28-37° C. All temperatures tested, resulted in similar efficiencies of fusion. Cells were incubated for 18 to 28 h post-transfection, before they were dissociated from wells using non-enzymatic cell dissociation solution following manufacturer's directions (Sigma, Catalog#C5789), resuspended in DMEM containing 10% FBS, 100 mg/ml rifampicin and 10 mM AraC, and mixed with target cells.

Huh-7.5 target cells were plated on fibronectin-coated (14 ug/ml; Sigma#F1141, following manufacturer's protocol) coverslips in a 24-well plate at ˜1.5×10⁵ cells/well and grown for ˜24 h at 37° C. Cells were infected with vaccinia virus vTF1.1 containing the T7 polymerase gene downstream of the viral late promoter. Viral inoculum contained in ˜200 mL serum-free DMEM was added to cells for ˜3 h at 37° C. One well containing cells transfected with T7IRES-GFP plasmid and an RFP expression plasmid was also infected with the same vaccinia virus to determine the efficiencies of transfection and infection. When at least 50% of cells in this well turned green, the transfection and infection processes were judged to be at acceptable levels for the assay to work efficiently. Viral inoculum was removed by washing cells with DMEM containing 10% FBS, 100 mg/ml rifampicin and 10 mM AraC. Effector cells were placed on top of target cells, ˜5×10⁵ effector cells/well in 24-well plates, and co-cultured for 4 to 6 h at 37° C. To initiate fusion, medium was removed and replaced with fusion buffer (135 mM NaCl, 15 mM Sodium Citrate, 10 mM MES, 5 mM HEPES, 1 mM EDTA, (made fresh from 10× stock and adjusted to the desired pH between 4.8 and 7.0 using HCl) for ≦1 min (9). The low pH buffer was replaced with DMEM containing 10% FBS, 100 mg/ml rifampicin and 10 mM AraC. After incubating the cells for 12 (range 5-18) h at 37° C., cells were examined by fluorescence microscopy using filters with emission spectra of 510-560 nm. Green multinucleated cells were counted in each well.

Modifications to the assay involved testing different constructs for expression of E1 and E2 proteins and various cell types as target cells (described in Cells, expression constructs and virus section). HCV antisera were pooled from over 25 patients with high titers of anti-HCV antibodies. Peptides “SAYQ” (amino acid sequence: SAYQVRNSSGLYHVTNDC (SEQ ID NO:3)) and “SSGLY” (SSGLYHVTNDCPNSSIVY (SEQ ID NO:4)) corresponding to amino acids 190 to 207 and 197 to 214 respectively, in E1 (WO 2004/044220 A2) were synthesized and purified by HPLC (NeoMPS Inc., San Diego, Calif.). Antisera and peptides were added to the co-culture in serum-free DMEM, at different concentrations, and incubated at 4° C. for ˜45-90 min immediately before the addition of low pH buffer for fusion. Subsequent incubations, before scoring for fusion, were without antisera or peptides.

Generation of a Library of Mutations in HCV E1 and E2 Sequences:

15-nucleotide insertional mutations were created in a plasmid derived from pcDNA3.1-HE1E2, using TnsABC transposase (New England Biolabs, Beverly, Mass.). To facilitate the mutagenesis, two PmeI restriction endonuclease sites located in non-coding regions of the plasmid were first altered using site-directed mutagenesis (Quikchange, Stratagene). This new construct, pHE1E2D2Pme, behaved like the original construct in fusion and pseudotyping assays. Mutations are denoted by the position of the amino acid just N-terminal to the insertion, with 1 being the first methionine of the C protein. Mutations reported in this study, along with their insertional sequences are as follows: 384-TLFKQ (SEQ ID NO:5) (corresponding to the insertion of these five amino acids at residue 216 of SEQ ID NO:2), 388-TGVST (SEQ ID NO:6) (corresponding to the insertion of these five amino acids at residue 220 of SEQ ID NO:2), 394-HTVST (SEQ ID NO:7) (corresponding to the insertion of these five amino acids at residue 226 of SEQ ID NO:2), 545-LCLNT (SEQ ID NO:8) (corresponding to the insertion of these five amino acids at residue 377 of SEQ ID NO:2), 548-WCLNN (SEQ ID NO:9) (corresponding to the insertion of these five amino acids at residue 380 of SEQ ID NO:2), 722-FLFKQ (SEQ ID NO: 10) (corresponding to the insertion of these five amino acids at residue 554 of SEQ ID NO:2), and 735-WCLNM (SEQ ID NO:11) (corresponding to the insertion of these five amino acids at residue 567 of SEQ ID NO:2).

Immunofluorescence:

Indirect immunofluorescence was performed mostly as described (Flint, M. et al., (1999) J Virol 73:6782-90). To determine whether mutations in E2 resulted in envelope protein expression, 293T cells growing in 6-well dishes were transfected with constructs expressing mutant proteins. Constructs expressing wild type proteins served as controls. 24 h after transfection, cells were plated onto fibronectin-coated coverslips, and another 24 h later, fixed with paraformaldehyde (4%). Half the set was permeabilized using 0.1% Triton X100 in PBS, while the other half was left untreated to visualize surface expression of E1 and E2 proteins. Non-specific binding was blocked by incubating cells with 10% goat serum. Human sera pooled from ˜25 patients with high titers of antibodies to HCV was used as primary antibody. Alexa Fluor 594-conjugated goat anti-human IgG was the secondary antibody (Invitrogen). Staining was visualized by fluorescence microscopy using a filter with emission spectra of 590-650 nm. For visualizing expression of envelope proteins in the fusion assay, cells were fixed and permeabilized as above, and stained with H52, a conformation-independent antiE2 mAb (Cocquerel, L. et al., (1998) J Virol 72:2183-91), and Alexa Fluor 594-conjugated goat anti-mouse IgG1 as primary and secondary antibodies respectively. For visualizing VSV-G expression in controls used for fusion, rabbit VSV antiserum (Lee Biomolecular Research Inc. #06141) was the primary antibody and Alexa Fluor 546-conjugated goat anti-rabbit IgG, the secondary antibody.

Generating Pseudoparticles Using Mutants in HCV E1 and E2 Proteins:

Subconfluent 293T cells in 10 cm dishes were co-transfected with 2 mg pCMVdelR8.91, 2 mg pCSRW, and 5 mg pcDNA3.1-HE1E2 (containing sequences for either wild type or mutant E1 and E2 proteins). Supernatant was collected at 48 h post-transfection, filtered through a 0.45 mm Whatman filter and assayed for reverse transcriptase (RT) activity to determine the amount of virions contained (Telesnitsky, A. et al., (1995) Methods Enzymol 262:347-62). Assays were quantified on a PhosphorImager (Molecular Dynamics) using ImageQuant (Amersham Biosciences). Preparations with equal amounts of RT activity were used to transduce Huh-7.5 target cells that were plated in 24-well plates (1×10⁵ cells/well) 24 h prior to transduction. Inoculum of pseudoparticles in serum-free DMEM was added to wells for 3 h at 37° C. before adding serum-containing medium. Medium was again replaced 24 h post-transduction, and cells were incubated at 37° C. for an additional 72-96 h. Transduction efficiency was determined by fluorescence microscopy using filters with emission spectra of 590-650 nm.

Results

Cell-Cell Fusion Assay Using HCV Envelope Proteins:

An assay was devised where fusion of two cell-types depended on the presence of functional HCV envelope proteins on the surface of one cell-type, HCV receptors on the other, and a fluorescent reporter system split between the two cell types. The scheme for this fusion assay is shown in FIG. 2. Due to their high transfection efficiencies, 293T cells were used to express HCV envelope proteins. Huh-7.5 liver cells were used as target cells since they permit entry of HCV pseudoparticles (Hsu, M. et al., (2003), Proc Natl Acad Sci USA 100:7271-6.), and must therefore express the HCV receptors. Co-cultivation of the cells allowed HCV envelope proteins on the surface of one cell-type to bind receptors on the other. Since fusion of many flaviviruses is thought to occur in endosomes, the pH of the co-culture was transiently lowered to pH 5.0. Many cells fused with each other, resulting in syncytia that fluoresced green due to expression of the reporter. For the reporter, 293T cells contained the GFP sequence downstream of a T7 promoter, and Huh-7.5 cells expressed high levels of T7 polymerase from vaccinia virus via the late promoter (details in materials and methods).

When the cytoplasm of the two cell types mixed as a result of fusion, green syncytia were formed (FIG. 3B, panel 1). Control cells transfected with an empty vector showed few, very faint green cells due to baseline expression from the T7 promoter in the absence of T7 polymerase (FIG. 1B, panel 2). Cells expressing viral envelope proteins that were exposed to an identical buffer, but at pH 7.0, also showed very few green cells (not shown). To confirm that fusion was dependent on the presence of HCV envelope proteins on the cell surface, cells were fixed, permeabilized and subjected to indirect immunofluorescence using antibodies to HCV envelope proteins. Each syncytium showed high levels of expression of HCV envelope proteins, further evidence that the large, green multinucleated cells specifically resulted from fusion mediated by HCV E1 and E2 proteins. The positive control for fusion consisted of 293T cells expressing VSV-G protein, which resulted in characteristic large syncytia at pH 5.0, but not at pH 7.0, and these stained with anti-VSV-G antibodies but not with anti-HCV antibodies (FIG. 3B, panel 3).

In contrast to the giant syncytia seen with VSV-G, HCV syncytia were small and typically contained 2-4 nuclei (see FIG. 3C for several examples). Thus a typical fusion experiment with VSV-G resulted in a single giant syncytium, inclusive of every cell in the well, while fusion with HCV E1 and E2 resulted in several hundred, discrete syncytia. There are at least two differences between VSV-G and HCV envelope proteins that could account for the observed differences in fusion. The phosphatidylserine receptor for VSV-G is present in every cell, allowing each cell to be included in the syncytium. By contrast, HCV receptors are only present on target cells, thus cells expressing E1 and E2 need to be directly adjacent to target cells for fusion to occur. More importantly, VSV-G protein is expressed at high levels on the surface of cells, whereas HCV E1 and E2 contain ER retention signals, and only a small fraction of these proteins is transported to the cell surface.

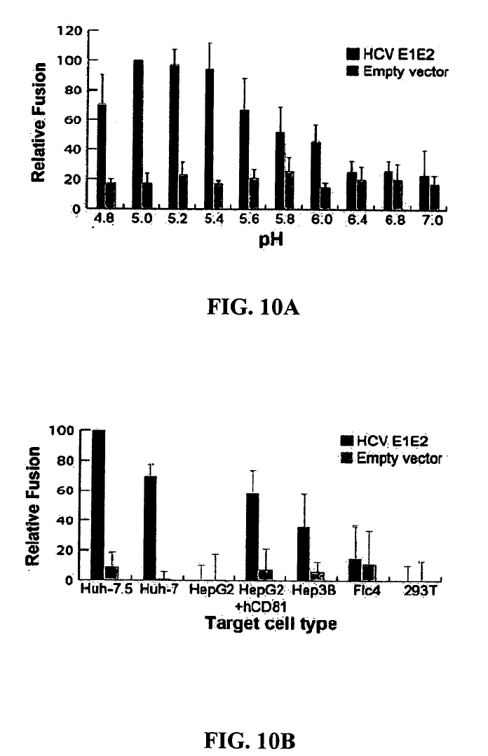

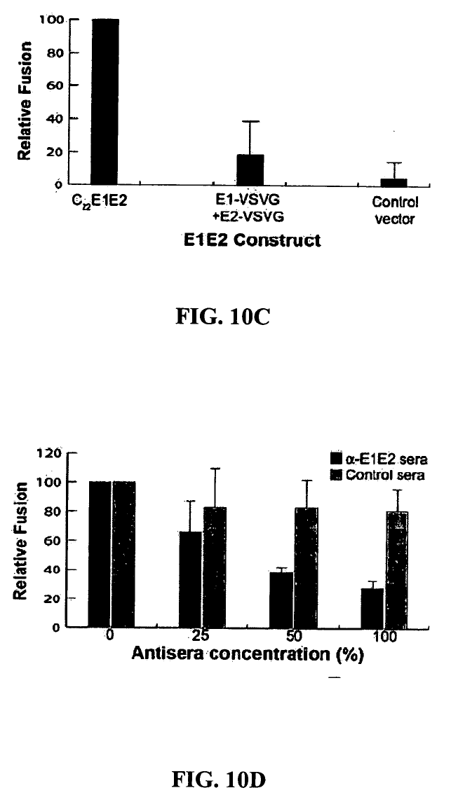

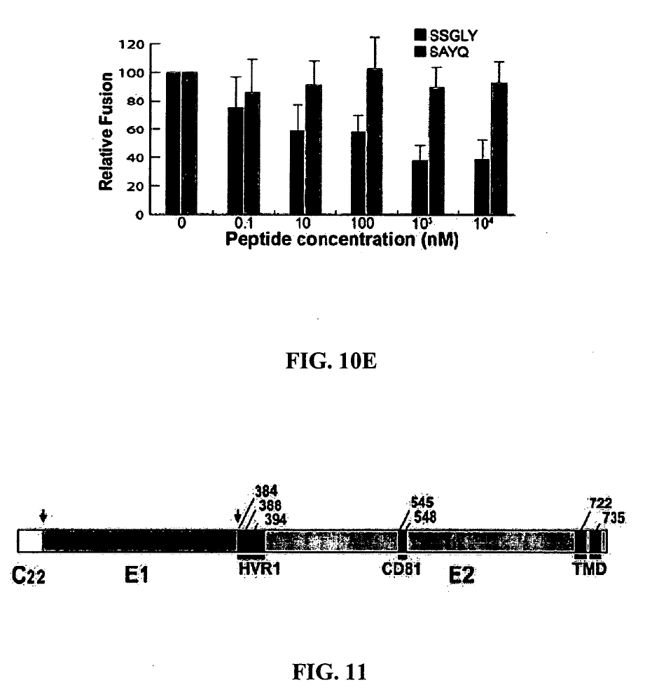

To investigate if syncytium formation depended on levels of fusion protein expression, each of the fusion experiments were followed with detection of E1 E2 expression levels by immunofluorescence (central images in FIG. 3B). Cells were permeabilized to visualize total cell-associated envelope proteins. In most cases, only cells that produced abundant amounts of E1 and E2 formed syncytia. Moderate to low levels of E1 and E2 expression often resulted in the cells remaining single. This indicated that high amounts of surface E1 and E2 might be important for efficient fusion. Even though the number of syncytia resulting from HCV fusion constituted only 1-2% of cells, this number was consistently 5-10 fold above the number of green cells seen at neutral pH, or at low pH with empty vectors (see FIG. 10). Furthermore, the use of a fluorescent reporter, viz. GFP, allowed unequivocal identification of E1 and E2 mediated syncytia, despite their smaller size.

Characterization of Fusion Mediated by HCV Envelope Proteins:

pH of Fusion. To determine the pH optimum for fusion mediated by HCV E1 and E2 proteins, the pH of the buffer used for fusion was varied, from pH 4.8 to pH 7.0 (FIG. 10A). Fusion was most efficient at pH 5.0 to 5.4, was progressively less efficient as the pH was raised, and was at background levels at pH 7.0. This confirmed that HCV, like other flaviviruses, requires low pH to fuse, and that the pH optimal for HCV fusion might be attained in the late endosome. Fusion appeared to be less efficient at pH 4.8, but that was likely due to decreased cell viability at this extreme pH.

Target cell specificity. Several human hepatocyte- and non-hepatocyte derived lines were tested for efficiency of HCV envelope protein mediated fusion (FIG. 10B). Fusion occurred with maximal efficiency with Huh-7.5 cells. Huh-7 cells were also efficient, while Hep3B cells fused with modest efficiency. HepG2 cells did not support fusion, which is corroborated by the finding that they do not support transduction with HCV pseudoparticles. However, HepG2 cells expressing the human CD81 receptor, one of the HCV co-receptors, fused efficiently. Another hepatocytic line, FLC4, and non-hepatocytic human lines such as 293T supported little to no fusion. All cell lines supported efficient fusion with VSV-G protein (not shown).

Envelope protein constructs. Fusion efficiency of HCV envelope proteins expressed from different constructs was also tested (FIG. 10C). E1 and E2 proteins from genotype 1a and 1b, the two most prevalent genotypes of HCV in the US, were both efficient at cell-cell fusion. Best results were obtained when E1 and E2 proteins were synthesized as a polyprotein. The C protein contains a hydrophobic signal sequence at its C-terminus that translocates the E1 protein into the ER lumen (42). C22E1E2 (see SEQ ID NO:2) expressed the terminal 22 amino acids of C, and the entire sequences of E1 and E2 proteins from HCV genotype 1a, and was very efficient for fusion. Similar constructs from genotype 1b were also tested, containing either the terminal 60 amino acids of the C protein or the entire C protein. Each fused efficiently (data not shown). Native E1 and E2 proteins (as expressed from each of these constructs) are known to be mostly retained in the ER, and this was confirmed by immunofluorescence.

It was reasoned that higher surface expression might result in more frequent fusion events and perhaps larger syncytia. Chimeric E1 and E2 proteins were tested that contained: (a) the ectodomains of HCV E1 and E2 proteins (not full length E1 and E2), and (b) the signal sequences, transmembrane domains and cytoplasmic tails of VSV-G protein. The two envelope proteins were on different constructs, and were introduced into cells in equimolar amounts. No HCV C protein sequences were present. Lacking signals for ER retention, the chimeric envelope proteins efficiently reach the cell surface, which was verified by immunofluorescence. These chimeric proteins were previously reported to mediate fusion in a different cell-cell fusion assay (Takikawa, S. et al., (2000) J Virol 74:5066-74). However, as studied herein, fusion with these chimeric proteins was not robust or reproducible, even when the assay was performed with different cell-types, different pH, at different times following transfection or infection (to test different levels of protein expression), or by utilizing constructs, cell lines and reporter used in the original assay.

Removal of the TM domains of E1 and E2 results in loss of both ER retention signals and dimerization sequences. Replacement with VSV-G TM domains allows efficient transport to the cell surface, suggesting that the chimeric proteins are not grossly misfolded, but fails to provide a dimerization domain, since VSV-G is a trimer. The absence of this dimerization domain or subtle differences in folding of the chimeric proteins might result in absence of fusion activity. Taken together, the data provided herein suggest that the envelope proteins are most efficient at fusion when expressed in their native conformation. The presence of a portion of the C-terminus of the C protein is essential for correct proteolytic processing of the E1 protein, and inclusion of this region appeared to strongly enhance fusion in the assays herein.

Fusion inhibitors. The specificity of envelope protein requirement for fusion was next tested by attempting to inhibit fusion using anti-HCV antibodies (FIG. 10D), or by using peptides derived from sequences in the envelope proteins (FIG. 11E). Human sera pooled from patients that had high levels of antibodies to HCV inhibited fusion by over 70% when bound to cells for an hour before treating cells with pH 5.0 fusion buffer. It is possible that the low pH treatment resulted in loss of some antibody binding, and therefore less than complete inhibition. Under the same conditions, control human sera had no effect. Neither sera affected VSV-G mediated fusion (not shown). Various peptides derived from sequences in HCV E1 and E2 have been demonstrated to inhibit infection with HCV pseudoparticles (WO 2004/044220 A2). The effect of two such peptides on fusion when added to cells at various concentrations for an hour before fusion was examined (FIG. 10E). A peptide corresponding to the sequence in E1 protein between amino acids 197 and 214 of the HCV polyprotein (SSGLY (SEQ ID NO:12)) inhibited fusion by ˜40% when added to cells at concentrations of 10 nM or above. Another peptide (SAYQ (SEQ ID NO:13)) corresponding to partly overlapping sequences (amino acids 190 to 207) had little effect on fusion even at concentrations up to 10 mM. This effect of peptides corresponds to reports that the peptide SSGLY inhibits transduction with pseudoparticles, while SAYQ does not. The assay thus provides an effective and simple means to screen for anti-HCV compounds that specifically inhibit HCV entry into cells.

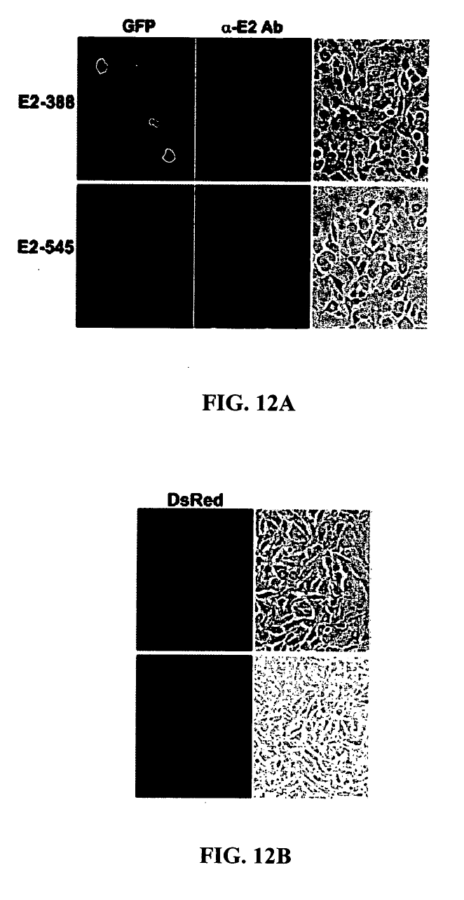

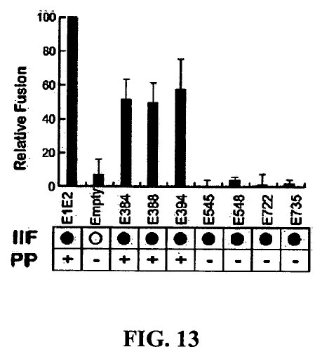

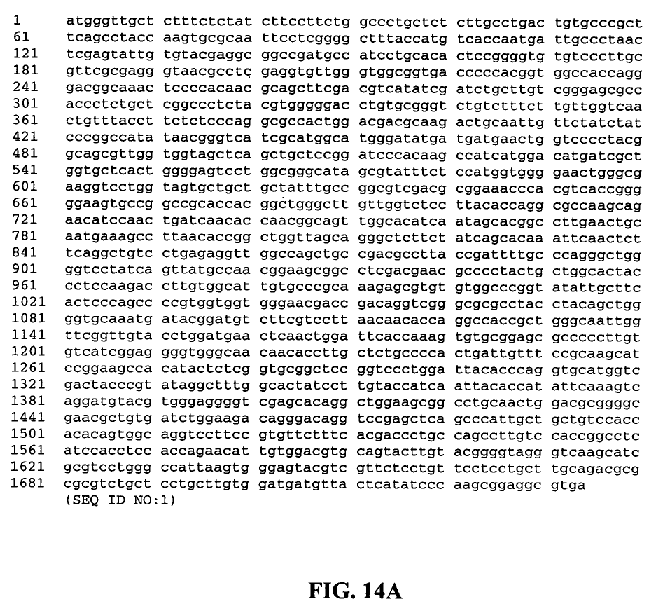

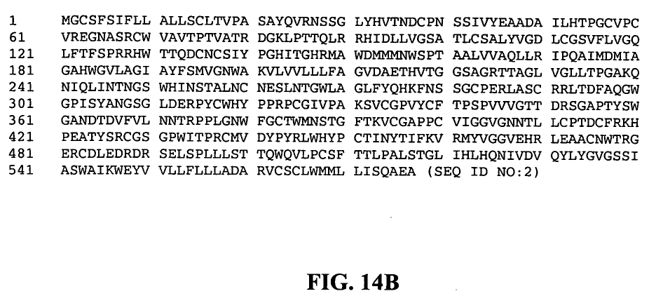

Generation and functional analysis of mutations in HCV E1 and E2 sequences: Using a Tn7-derived transposase, a set of insertional mutations in the sequences coding for E1 and E2 proteins from HCV genotype 1a was generated. Each mutant contained a single insertion of 15 nucleotides (coding for 5 amino acids—see SEQ ID NOs:5-11 for sequences that were inserted) at a random location. To begin to test the assay for measuring fusion efficiency of envelope protein mutants, seven mutants were chosen from a library whose locations might predict effects on fusion (FIG. 11). It was reasoned that the CD81 binding site might not tolerate insertions, since CD81 is necessary for HCV entry. Similarly, the transmembrane domains, with their roles in protein folding and dimerization may also be essential for fusion. In contrast, the hypervariable region might tolerate 5 amino acid insertions, since it varies strikingly among the different genotypes of HCV, and even during the course of HCV infection in a given patient. As expected, it was found that insertional mutations in the CD81 binding site (one at amino acid 545 and the other at amino acid 548 of the HCV polyprotein—see supra for residue numbering corresponding to SEQ ID NO:2) prevented fusion, while insertions in the hypervariable region (at amino acids 384, 388 and 394) permitted fusion activity at levels that were approximately 60% of those obtained with wild type envelope proteins (FIGS. 12A and 13). Insertions in the E2 transmembrane domains (at amino acids 722 and 735) also prevented fusion, as expected. The presence of 3 separate fusion-competent mutants in the HVR1 demonstrated that this region could tolerate insertions of 5 amino acids without significant loss of function.

To determine whether the lack of fusion activity was due to a lack of mutant envelope protein expression, envelope protein levels were examined for each mutant by indirect immunofluorescence. Polyclonal anti-HCV antisera was used to ensure that all mutant proteins were effectively visualized. Both permeabilized and intact cells were stained to determine amounts of envelope proteins in the entire cell and those at the cell surface only. It was found that all mutants with read-through insertions were indistinguishable from wild type by immunofluorescence, both in the amounts of protein produced and their intracellular location (FIG. 12A and FIG. 13). However, it was not determined whether any mutants failed to fuse because the amount of protein expressed on the cell surface was too low. Both E1 and E2 proteins contain signals for retention in the endoplasmic reticulum (ER). It is thought that flaviviral viral cores bud into the ER, and the virus is transported outside the cell via the secretory pathway. Flow cytometry shows that a very small fraction of total protein escapes ER-retention and is expressed on the cell surface. Reliable measurements of an additional decrease from such a small fraction for any of the mutants were not obtained.

The cell-cell fusion assay discussed in this Examples was corroborated with a different method that measures HCV envelope protein-dependent viral entry. Pseudoparticles that contain a retroviral core and HCV envelope glycoproteins were recently described (Hsu, M. et al., (2003) Proc Natl Acad Sci USA 100:7271-6; Bartosch, B. et al., (2003) J Exp Med 197:633-42). Transduction with these pseudoparticles depends on the presence of functional HCV envelope proteins. Fusion activity of mutants tested above was compared with the infectivity of pseudoparticles containing mutant HCV proteins. These pseudoparticles contained mutant HCV envelope proteins and a gene for the fluorescent marker DsRed in the plasmid containing the retroviral packaging sequence (described in Methods). Pseudoparticles were harvested and titrated by measuring activity of reverse transcriptase. Equal amounts of inoculum from each mutant were used to transduce Huh-7.5 cells. The highest inoculum, made with wild type E1 and E2 proteins, efficiently transduced Huh7.5 cells, as monitored by fluorescence microscopy. Control transductions with empty vector, or with the chimeric HCV E1-VSVG and E2-VSVG resulted in rare and faint red cells (not shown).

Infectivity of pseudoparticles generated using the envelope protein mutants (FIGS. 12B and 13) was tested. Mutants that were fusion-competent also transduced cells efficiently. Mutants that could not fuse were also unable to transduce. This further confirms that the cell-cell fusion assay measures a process that is dependent on HCV entry into cells.

Discussion

The cell-cell fusion assay described in this Example provides a simple, quantitative and versatile tool to study HCV mediated fusion. The advantages of this system come from its several features and includes (but is not limited to the following). (1) The ability to isolate the steps of binding and fusion from the rest of the viral lifecycle, which allows for focused study of these steps and the means for receptor identification. (2) The use of GFP as a reporter, which makes the assay highly sensitive and able to detect relatively rare events. By contrast, enzymatic reporters such as luciferase or β-galactosidase need lysis of cells and are better suited for studying systems where the majority of cells in the assay fuse. The fluorescent reporter further allows the possibility of high-throughput screens. The absence of biohazards that accompany studies using infectious viruses is especially relevant for HCV. The HCV isolate that successfully replicated in vitro (Zhong, J. et al., (2005) Proc Natl Acad Sci USA, 102 (26) 9294-9299; Lindenbach, B. D. et al., (2005), Science, 309 (5734):623-626; Wakita, T. et al., (2005), Nat Med 11:791-6) was very unusual in that instead of causing chronic hepatitis, it caused fulminant hepatic necrosis, a condition that is often rapidly fatal. Studying fusion of this unusually virulent isolate or making an extensive library of mutations in the corresponding infectious clone would require additional safety measures not necessary for the fusion assay of this Example. The assay works with at least genotypes 1a and 1b of HCV. This is in contrast to the in vitro replication systems that so far only work with an isolate from genotype 2a. The ability to study fusion with genotypes 1a and 1b is especially useful, since these genotypes are the most prevalent, are most likely to progress to chronic liver disease, as well as most resistant to antiviral therapy. Furthermore, the chronic sequelae of HCV, such as cirrhosis and hepatocellular carcinoma are related to the propensity of the virus to cause chronic inflammation. Fulminant hepatic necrosis in contrast, is an acute condition, and in cases that recover, is not followed by chronic liver disease.

The ability to inhibit fusion with antibodies and peptides offers an efficient means to screen for antivirals. (Bartosch, B. et al. (2003) J Exp Med 197:633-42.) The assay is fairly rapid, and can be scored within a few hours (as early as 4-6 h) after fusion has occurred—in contrast to transduction with pseudotyped particles where a signal indicating viral entry is seen several days (typically 3 to 5 d) after transduction, when sufficient amounts of retroviral protein synthesis has occurred.

A fusion assay utilizing chimeric E1-VSVG and E2-VSVG proteins, HepG2 cells as target cells and luciferase as reporter, was previously reported (Takikawa, S. et al. (2000) J Virol 74:5066-74). Despite repeated attempts, this assay was unsuccessful, either as originally reported, or by using different target cells and GFP as reporter. The great number of variations attempted allowed the conclusion that at least three factors were crucial for the fusion assay of this Example to work. The use of a sensitive reporter like GFP was mentioned above. The use of native HCV envelope protein constructs instead of chimeras was also important. The dimerization domains of HCV envelope proteins are in the transmembrane regions of E1 and E2. Substitution of this region with the corresponding region from VSV-G not only removes the native dimerization signal but also adds a portion of a protein that is normally a trimer. It is possible that the HCV-VSVG chimeric proteins do not form dimers, or do not assume conformations necessary for fusion, leading to a lack of robustness in fusion assays that employ them. This is further corroborated by data from chimeric HCV-influenza HA proteins, which also do not fuse. Finally, various human liver cell lines were tested and it was found that Huh-7.5 cells, and the related Huh-7 cells, gave the best fusion. Both of these cells express high levels of CD81, which is known to be necessary, though not sufficient, to mediate entry of pseudoparticles. HepG2 cells, which express little CD81 receptor on their surface, did not permit fusion in the assay, in contrast to the previously reported HCV fusion assay that utilized HCV-VSVG chimeric proteins.

There have been no mutational analyses of HCV envelope proteins that test fusion yet. An extensive library of mutants in E1 and E2 were generated, and the fusion assay of this Example provides a convenient method to study them. Initial analysis of a few mutants from this library has revealed a region that can tolerate insertions of 5 amino acids in at least 3 discrete positions. The data suggests that the HVR1 might be especially useful to generate epitope-tagged virus for viral entry studies. The recovery of functional mutants also suggests that not all insertional mutants into the HCV E1 and E2 result in folding or transport defects. An analysis of a comprehensive library of mutants is likely to provide a functional map of the HCV envelope proteins.

In summary, the fusion assay allowed one to define important characteristics of HCV fusion. For the first time, the measurement of pH of HCV fusion was conducted. It was found that the envelope proteins needed to be in their native conformation for effective function, and this was aided by including at least 22 amino acids from the signal sequence of E1 located at the C terminus of C protein in the expression construct. The extent of fusion permitted by various cell lines suggested levels of HCV receptor(s) expression in different cell lines. Such information is necessary for using this assay, or other cell-based assays, to identify the HCV receptor(s). A region in the E2 protein was also identified that could tolerate insertions, and demonstrated how the assay could be used for screening, or selecting for, functional mutants in HCV envelope proteins. It was shown that fusion could be effectively inhibited by antibodies and peptides, offering a useful method to screen for antiviral agents. Current anti-HCV therapy consisting of ribavirin and interferon-α is effective in less than half of all treated patients. There is a need for drugs that target other steps in the viral lifecycle. The assay provides for easy application of high-throughput procedures, which would accelerate the discovery of new antiviral agents.

What is claimed:

1. A method for propagating infectious hepatitis C virus (HCV) particles in a monolayer or suspension cell culture which comprises:
(a) (i) infecting a cell with a virus particle comprising an HCV E1 protein and an HCV E2 protein or (ii) transfecting a cell with one or more nucleic acids comprising a HCV nucleic acid, wherein the one or more nucleic acids encode a virus; and
(b) incubating the cell at a temperature from about 20° C. to about 34° C. in an appropriate cell culture system, wherein the cell culture system is not a bioreactor, thereby propagating infectious HCV particles.

2. The method of claim 1, wherein the HCV nucleic acid comprises a sequence encoding a HCV E1 protein and a HCV E2 protein.

3. The method of claim 2, wherein the sequence encodes a full-length HCV E1 protein and a full-length HCV E2 protein.

4. The method of claim 1, wherein the virus particle comprises a full-length HCV E1 protein and a full-length HCV E2 protein.

5. The method of claim 1, wherein the one or more nucleic acids comprise a full-length HCV genome.

6. The method of claim 1, wherein the bioreactor is a radial-flow bioreactor.

7. The method of claim 1, wherein the cell culture system comprises a monolayer.

8. The method of claim 1, wherein the cell culture system comprises a suspension tissue culture system.

9. The method of claim 1, wherein the cell comprises a hepatocyte cell, a monkey kidney cell, a porcine kidney cell, a baby hamster kidney cell, a murine macrophage cell, a human macrophage cell, a human peripheral blood leukocyte, a human adherent macrophage, an embryonic cell, a stem cell, or a transformed cell.

10. The method of claim 9, wherein the cell comprises a primary cell or a cell line.

11. The method of claim 10, wherein the cell line comprises a human hepatoma cell-line.

12. The method of claim 11, wherein the human hepatoma cell line comprises Huh-7, Huh-7.5, PLC/PRF-5, FLC4, Hep3B, HepG2 or HepG2-CD81.

13. The method of claim 1, wherein the cell comprises Huh-7.

14. The method of claim 1, wherein the virus particle comprises a wild-type particle or a pseudotyped particle.

15. The method of claim 1, wherein the one or more nucleic acids comprise a full-length wild-type virus genome, a hepatitis C virus subgenome, or a combination of expression constructs comprising hepatitis C virus envelope glycoproteins and retroviral or lentiviral core proteins.

16. The method of claim 1, wherein the temperature is from about 26° C. to about 30° C.

17. A method for identifying a drug that inhibits HCV propagation comprising:
(a) (i) infecting a cell with an HCV particle or (ii) transfecting a cell with one or more nucleic acids comprising an HCV nucleic acid, wherein the one or more nucleic acids encode a virus;
(b) contacting the cell with a drug in a cell culture system;
(c) incubating the cell culture system at a temperature from about 20° C. to about 34° C.; and
(d) assaying the cell culture supernatant for the presence of infectious HCV particles, wherein the absence or reduction of infectious HCV particles compared to a supernatant from an uninfected or non-transfected cell culture, identifies the drug as a drug that inhibits HCV propagation.

18. A method for identifying a mutation that modulates HCV infection of cells comprising:
(a) (i) infecting a cell with an HCV particle comprising a genome having at least one mutation or (ii) transfecting a cell with one or more nucleic acids comprising an HCV genome having at least one mutation;
(b) incubating the cell in a cell culture system at a temperature from about 20° C. to about 34° C.; and
(c) assaying the cell culture supernatant for the presence of infectious HCV particles, wherein the absence or reduction of infectious HCV particles compared to a supernatant from a cell culture infected or transfected with an unmutated HCV genome indicates that the mutation modulates HCV infection of cells.

19. The method of claim 17 or claim 18, wherein the cell comprises a liver cell expressing CD81, HLDLr and hSR-B1.

20. The method of claim 19, wherein the liver cell comprises a hepatoma or a hepatocellular carcinoma.

21. The method of claim 20, wherein the hepatoma or hepatocellular carcinoma comprises Huh-7, PLC/PRF/5, Hep3B or HepG2-CD81.

22. A method for detecting HCV E1 and E2 mediated cell fusion comprising:
(a) co-culturing in a cell culture system an effector cell with a target cell, wherein the effector cell comprises an HCV E1 protein and an HCV E2 protein that are displayed on the effector cell surface;
(b) incubating the cell culture system at a temperature from about 20° C. to about 34° C.; and
(c) assaying the co-culture for syncytium formation, thereby detecting HCV E1 and E2 mediated cell fusion.

23. A method for detecting HCV E1 and E2 mediated cell fusion comprising:
(a) co-culturing in a cell culture system an effector cell with a target cell, wherein the effector cell comprises an HCV E1 protein and an HCV E2 protein that are displayed on the effector cell surface, wherein the E1 protein comprises an E1 ectodomain and an E1 transmembrane domain, and wherein the E2 protein comprises an E2 ectodomain and an E2 transmembrane domain;
(b) transiently incubating the cell culture system at a pH from about 5.0 to about 5.4; and
(c) assaying the co-culture for syncytium formation, thereby detecting HCV E1 and E2 mediated cell fusion.

24. A method for detecting HCV E1 and E2 mediated cell fusion comprising:
(a) co-culturing in a cell culture system an effector cell with a target cell, wherein the effector cell comprises:
(i) a trigger that activates a reporter,
(ii) an HCV E1 protein displayed on the effector cell surface, and
(iii) an HCV E2 protein displayed on the effector cell surface,
and wherein the target cell comprises the reporter, wherein the E1 protein comprises an E1 ectodomain and an E1 transmembrane domain, and wherein the E2 protein comprises an E2 ectodomain and an E2 transmembrane domain;
(b) transiently incubating the cell culture system at a pH from about 5.0 to about 5.4; and
(c) assaying the co-culture for reporter activity, wherein detection of reporter activity indicates HCV E1 and E2 mediated cell fusion.

25. The method of claim 24, wherein step (b) further comprises incubating the cell culture system at a temperature from about 20° C. to about 34° C.

26. A method for identifying a polypeptide involved in HCV E1 and E2 mediated cell fusion comprising:
(a) co-culturing in a cell culture system an effector cell with a target cell, wherein the effector cell comprises an HCV E1 protein and an HCV E2 protein that are displayed on the effector cell surface, and wherein the target cell comprises:
(i) a polypeptide encoded by a library vector that is displayed on the target cell surface,
(ii) a CD81 protein displayed on the target cell surface, and
(b) incubating the cell culture system at a temperature from about 20° C. to about 34° C.; and
(c) detecting increased syncytium formation as compared to a control co-culture wherein the target cell does not comprise the library vector, wherein detection of increased syncytium formation indicates that the polypeptide is involved in HCV E1 and E2 mediated cell fusion.

27. A method for identifying a polypeptide involved in HCV E1 and E2 mediated cell fusion comprising:
(a) co-culturing in a cell culture system an effector cell with a target cell,
wherein the effector cell comprises:
(i) a trigger that activates a reporter,
(ii) an HCV E1 protein displayed on the effector cell surface, and
(iii) an HCV E2 protein displayed on the effector cell surface;
and wherein the target cell comprises:
(i) the reporter,
(ii) a polypeptide encoded by a library vector that is displayed on the target cell surface,
(iii) a CD81 protein displayed on the target cell surface,
(b) incubating the cell culture system at a temperature from about 20° C. to about 34° C.; and
(c) assaying the co-culture for increased reporter activity as compared to a control co-culture wherein the target cell does not comprise the library vector, wherein detection of increased reporter activity indicates that the polypeptide is involved in HCV E1 and E2 mediated cell fusion.

28. The method of claim 26 or 27, wherein the target cell further comprises an HLDLr protein displayed on the target cell surface or a SR-B1 protein displayed on the target cell surface.

29. A method for identifying a mutation that inhibits HCV E1 and E2 mediated cell fusion comprising:
(a) co-culturing in a cell culture system an effector cell with a target cell capable of HCV E1 and E2 mediated cell fusion, wherein the effector cell comprises:
(i) a trigger that activates a reporter,
(ii) an HCV E1 protein displayed on the effector cell surface, and
(iii) an HCV E2 protein displayed on the effector cell surface, wherein at least the E1 protein or the E2 protein contains a mutation, and wherein the target cell comprises a the reporter;
(b) incubating the cell culture system at a temperature from about 20° C. to about 34° C.; and
(c) assaying the co-culture for decreased reporter activity as compared to a control co-culture wherein E1 and E2 do not contain the mutation, wherein detection of decreased reporter activity indicates that the mutation inhibits HCV E1 and E2 mediated cell fusion.

30. A method for identifying a molecule that inhibits HCV E1 and E2 mediated cell fusion comprising:
(a) co-culturing in a cell culture system an effector cell with a target cell capable of HCV E1 and E2 mediated cell fusion, wherein the effector cell comprises:
(i) a trigger that activates a reporter,
(ii) an HCV E1 protein displayed on the effector cell surface, and
(iii) an HCV E2 protein displayed on the effector cell surface, and wherein the E1 protein comprises an E1 ectodomain and an E1 transmembrane domain, and wherein the E2 protein comprises an E2 ectodomain and an E2 transmembrane domain;
(b) adding a molecule to the cell culture system;
(c) transiently incubating the cell culture system at a pH from about 5.0 to about 5.4; and
(d) assaying the co-culture for decreased syncytia formation as compared to a control co-culture that is not exposed to the molecule, wherein detection of decreased syncytia formation indicates that the molecule inhibits HCV E1 and E2 mediated cell fusion.

31. A method for identifying a molecule that inhibits HCV E1 and E2 mediated cell fusion comprising:
(a) co-culturing in a cell culture system an effector cell with a target cell capable of HCV E1 and E2 mediated cell fusion, wherein the effector cell comprises:
(i) a trigger that activates a reporter,
(ii) an HCV E1 protein displayed on the effector cell surface, and
(iii) an HCV E2 protein displayed on the effector cell surface, wherein the E1 protein comprises an E1 ectodomain and an E1 transmembrane domain, wherein the E2 protein comprises an E2 ectodomain and an E2 transmembrane domain, and wherein the target cell comprises a repressor of the reporter;
(b) adding a molecule to the cell culture system;
(c) transiently incubating the cell culture system at a pH from about 5.0 to about 5.4; and
(d) assaying the co-culture for decreased reporter activity as compared to a control co-culture that is not exposed to the molecule, wherein detection of decreased reporter activity indicates that the drug inhibits HCV E1 and E2 mediated cell fusion.

32. The method of claim 30 or 31, wherein step (b) further comprises incubating the cell culture system at a temperature from about 20° C. to about 34° C.

33. The method of claim 29, 30, or 31, wherein the target cell capable of HCV E1 and E2 mediated cell fusion comprises cell-surface expression of CD81.

34. The method of claim 29, 30, or 31, wherein the target cell capable of HCV E1 and E2 mediated cell fusion comprises cell-surface expression of CD81 and SR-B1.

35. The method of claim 29, 30, or 31, wherein the target cell capable of HCV E1 and E2 mediated cell fusion comprises cell-surface expression of CD81 and HLDLr.

36. The method of claim 29, 30, or 31, wherein the target cell capable of HCV E1 and E2 mediated cell fusion comprises cell-surface expression of CD81, SR-B1 and HLDLr.

37. The method of any one of claims 22-31, wherein the effector cell comprises full-length HCV E1 and E2 proteins such that the proteins dimerize and the HCV E1 ectodomain and the HCV E2 ectodomain are displayed on the cell surface.
